(19) United States
(12) Patent Application Publication
(10) Pub. No.: US 2023/0183353 A1
(43) Pub. Date: Jun. 15, 2023

(54) FC VARIANT AND PREPARATION THEREOF

(71) Applicant: ZYDUS LIFESCIENCES LIMITED, Ahmedabad, Gujarat (IN)

(72) Inventors: Sanjeev Kumar MENDIRATTA, Ahmedabad, Gujarat (IN); Ramkrashan KASERA, Ahmedabad, Gujarat (IN); Arun Kumar SINGH, Ahmedabad, Gujarat (IN); Aashini PARIKH, Ahmedabad, Gujarat (IN); Pankaj KALITA, Ahmedabad, Gujarat (IN); Satish HANDA, Ahmedabad, Gujarat (IN); Anushree SHAH, Ahmedabad, Gujarat (IN); Heena PATEL, Ahmedabad, Gujarat (IN); Hardik PANDYA, Ahmedabad, Gujarat (IN); Vibhuti SHARMA, Ahmedabad, Gujarat (IN); Chirag PATEL, Ahmedabad, Gujarat (IN); Swagat SONI, Ahmedabad, Gujarat (IN); Narayani VYAS, Ahmedabad, Gujarat (IN)

(21) Appl. No.: 17/916,738

(22) PCT Filed: May 21, 2021
(86) PCT No.: PCT/IB2021/054423

§ 371 Date: Oct. 3, 2022

(30) Foreign Application Priority Data
May 21, 2020 (IN) 202021021451

(51) Int. Cl. C07K 16/28; A61P 7/00

(52) U.S. Cl. CPC C07K 16/283; A61P 7/00; C07K 2319/30; C07K 2317/70; C07K 2317/52; C07K 2317/92; C07K 2317/33; C07K 2317/41; A61K 2039/505

(57) ABSTRACT

The present invention relates to Fc variant protein and preparation thereof. Said Fc variant has altered binding affinity towards FcRn. Fc variant prepared according to the current invention can be used for making FcRn antagonist composition or can be used for making an Fc variant containing drug or molecule with altered effector function.

### FIELD OF THE INVENTION

The present invention relates to Fc variant protein and preparation thereof. Said Fc variant has altered binding affinity towards FcRn. Fc variant prepared according to the current invention can be used for making FcRn antagonist composition or can be used for making an Fc variant containing drug or molecule with altered effector function.

### BACKGROUND OF THE INVENTION

Neonatal Fc receptor (FcRn) is structurally homologous to the Major Histocompatibility Complex (MHC) Class I heterodimer molecule consisting of a type I transmembrane heavy chain that non-covalently associates with the soluble light chain, β2-microglobulin (β2m). β2m is essential for the proper folding, transport, and function of FcRn, as well as other MHC Class I homologs. The FcRn heavy chain contains three soluble extracellular domains (α1, α2, and α3), a single transmembrane helix, and a cytoplasmic tail. Unlike MHC Class I molecules, FcRn does not directly present antigens to T-cells due to point mutations on the top face of FcRn that disrupt peptide binding. The ability of FcRn to protect IgG from intracellular catabolism is the result of a specific, pH-dependent interaction with the Fc portion of IgG. IgG binds FcRn in a strictly pH-dependent manner at acidic (<6.5) but not neutral pH (>7) mediated by electrostatics between titratable histidine residues in the CH2-CH3 domains of IgG Fc and acidic residues on the α2-domain of FcRn. Binding is further stabilized by a series of hydrophobic interactions and hydrogen bonds between Fc and residues within the FcRn α2-domain and the β2m light chain N-terminus. One IgG molecule can simultaneously bind two FcRn molecules due to the homodimeric nature of IgG resulting in a high affinity interaction between FcRn and IgG at pH 6 due to increased avidity of interaction. The 2:1 interaction between FcRn and IgG is critical for their efficient binding, recycling, and transcytosis in the FcRn expressing cells ultimately leading to the preservation of the long serum persistence of IgG such that protective antibody molecules do not have to be repeatedly produced by B cells thereby providing efficient immunity against infections. Unfortunately this same interaction also contributes to the pathogenicity of those IgG molecules that are reactive toward a self-antigen leading to numerous autoimmune disorders. Mice deficient in FcRn are protected against IgG-mediated autoimmune diseases indicating that FcRn contributes, at least in part, to autoimmunity by maintaining the pathogenic IgGs in circulation longer. Because FcRn contributes to the serum persistence of IgG, therapeutics that block the IgG-FcRn interaction represent a potential treatment modality for IgG-mediated autoimmunity. In fact, high dose intravenous immunoglobulin (IVIg) therapy is FDA-approved for the treatment of IgG-mediated autoimmune diseases where it provides a therapeutic benefit in part by saturation of FcRn receptors, thereby increasing the catabolism of pathogenic IgG. However, IVIg therapy suffers from multiple problems. For example being a blood-derived product, it is susceptible to being contaminated with human pathogens such as HIV, HBV, HCV etc. and thus making the treatment of chronic disorders such as autoimmune diseases highly risky for the recipient patients. It is also expensive, since it requires IgG extraction from the plasma of many blood donors. Also, there is a concern of scalability because it is a human donor dependent product. Thus, novel recombinant approaches are the need of the hour to provide alternative safe, scalable and cost effective treatment options.

Monoclonal antibody based antagonists have been developed to inhibit the endogenous IgG-FcRn interaction. One approach is the use of monoclonal antibodies directed against FcRn working via the classical antibody: antigen binding mechanism. Examples of such antibodies are M281 and UCB7665 which bind human FcRn to inhibit IgG binding to FcRn and thereby accelerate the clearance of endogenous IgG. A mean reduction of endogenous IgG ranging between 25-80% was observed with M281 in a dose dependent manner. Moreover, single doses of M281 at 30 mg/kg or 60 mg/kg maintained serum IgG at 50% of baseline or below for 18 and 27 days, respectively (1). In healthy subjects, IgG concentrations in the serum were reduced by up to 50% at a single IV dose of 7 mg/kg of UCB7665. The observed reductions in serum IgG concentrations in the current study persisted for weeks, with maximal reductions achieved by days 7 to 10 and thereafter gradually returning to baseline by day 57 (2).

An alternative antibody engineering approach to reduce IgG levels in serum is to engineer the Fc-domain of IgG such that it has high affinity for FcRn at both pH 6 and pH 7.4. Such a molecule developed using the ‘Abdegs’ (i.e., antibodies that enhance IgG degradation) technology has been found to be effective in treating a murine model of arthritis at 25 to 50 fold lower dose than IVIg indicating that such IgG Fc-based potent antagonists of the IgG-FcRn interaction are an alternative therapeutic intervention in autoimmunity. This molecule, Efgartigimod, is in clinical development by the Netherlands-based biotech company, arGEN-X.

More recently, synthetic peptides that compete with IgG for binding to FcRn were identified from a phage library. A chemically optimized peptide dimer (SYN1436) that bound FcRn with sub nanomolar affinity at pH 6 and pH 7.4 was effective at increasing the clearance of exogenous IgG in mice as well as endogenous IgG in monkeys, but has not been evaluated in a therapeutic model of IgG-mediated autoimmunity. Alternatively, additional molecules that bind instead to Fc itself, at the CH2-CH3 domain interface to block the IgG-FcRn interaction, include a 13-amino acid cyclic peptide (FcIII) selected by phage display, a computationally designed IgG-Fc binding protein (FcBP6.1), and an endogenous Fc receptor, TRIM21. (3) Thus, the present invention provides an FcRn binder that solves various unmet needs such as, downregulation of the FcRn activity, increasing the circulating half-life of a variety of drugs, targeting of drugs and/or vaccines to specific cell types etc. The FcRn binder of the present invention can be used in treating a wide variety of diseases.

There is another class of molecules which targets immune-complex (IC) formation and immune-complex mediated FcγR mediated activation. This class of molecules are recombinant Fc multimers such as Pf-06755347 (GL-2045) and CSL730 (M230). These molecules are complex in terms of structure and may result into highly heterogenous population. These molecules are being developed for the treatment of autoimmune disease. Therefore, molecules targeting different mechanisms are under early clinical trial that are being developed as a replacement of intravenous immunoglobulin (IVIg) and subcutaneous immunoglobulin (SCIg) to avoid the dependence on the supply of human plasma and the large doses of product of up to 2 g/kg body weight needed for therapy. In this situation, it would be advantageous to get single drug that has combined effects of multiple mechanism of actions such as FcRn blockade, inhibition of FcγR activation and/or inhibition of full complement activation.

In one of the aspects, the current invention provides Fc variant molecule which is FcRn binder as well as FcγRs binders and may solve various needs together such as downregulation of the FcRn activity, increasing the circulating half-life of a variety of drugs, IC inhibition, inhibition of cytokine release mediated by IC formation, inhibition of phagocytosis mediated by FcγR activation. Preferably, the Fc variant molecule prepared according to the current invention provides one or more of the above said desired effects at a lesser dose as compared to the dose of IVIg.

### SUMMARY OF THE INVENTION

The current invention provides novel Fc variant with altered binding affinity towards FcRn, preferably higher binding affinity towards FcRn. In one of the aspects, the Fc variant of the current invention has altered binding affinity towards FcγRs, preferably higher binding affinity towards FcγRs. Preferably, the said Fc variant can be used to develop drug compositions with FcRn antagonist function or increased circulating half-life, or for targeting to specific cells and/or tissues. The present invention also provides method of making novel Fc variant. The Fc variant according to the present invention is further used in the preparation of a drug either for treating diseases where activity of FcRn is detrimental or for increasing the circulating half-life of a drug or for targeting the drug to certain cells or tissues.

### BRIEF DESCRIPTION OF FIGURES

FIG. 1: It depicts map of the vector used in library generation to make Fc variants of the present invention.

FIG. 2: It depicts map of the vector used for the generation of Fc monomer and Fc dimer of the present invention.

FIG. 3: It depicts map of the vector used for the generation of monoclonal antibody containing Fc monomer of the present invention.

FIG. 4: It depicts the results of experiment to determine the effect of Fc variant (Fc dimer) and IVIg on total IgG serum levels in wild-type mice.

FIG. 5: It depicts the results of experiment to determine the effect of Fc variant (Fc dimer) and IVIg on serum albumin levels in wild-type mice.

FIG. 6: It depicts results of experiment to determine the effect of Fc variant on ADCC activity.

FIG. 7: It depicts results of experiment to determine the effect of Fc variant on platelet count in a mouse model of acute immune thrombocytopenia (ITP).

### DEFINITIONS

The term “afucosylated” as used herein refers to a glycosylated protein with an N-linked glycan which lacks a core fucose molecule as described in U.S. Pat. No. 8,067,232, the contents of which is incorporated by reference herein in its entirety.

The term “amino acid modification” as used herein is an amino acid substitution, insertion, and/or deletion in a polypeptide sequence.

The term “amino acid substitution” or “substitution” as used herein is the replacement of an amino acid at a particular position in a parent polypeptide sequence with another amino acid. For example, the substitution T307N refers to a variant polypeptide, in this case an Fc variant, in which the threonine at position 307 is replaced with asparagine.

The term “antigen-binding molecule” according to the current invention refers to a protein that can bind to target antigen and comprising an “FcRn binding domain”. FcRn Binding domain according to the current invention is present in Fc protein, preferably, present in Fc variant of the current invention. Preferred “antigen-binding molecule” according to the present invention may include antibody or peptibody or fusion proteins comprising Fc variant of the present invention.

The term “antibody” as used herein includes whole antibodies and any antigen-binding fragments (i.e., “antigen-binding portion”). An “antibody” refers to a glycoprotein comprising at least two heavy (H) chains and two light (L) chains inter-connected by disulfide bonds, or an antigen-binding portion thereof. Each heavy chain is comprised of a heavy chain variable region (abbreviated herein as V_{H}) and a heavy chain constant region (Fc). The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hyper variability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells such as, NK cells, T cells, macrophages and dendritic cells etc.) and the first component (C1q) of the classical complement system.

The term “operatively linked” is intended to mean that a gene is ligated into a vector such that transcriptional and translational control sequences within the vector serve their intended function of regulating the transcription and translation of such gene.

The term “Kₐ” is the association rate of interaction between two molecules, whereas the term “K_{d}” is the dissociation rate of the interaction between two molecules. The term “K_{D}” is an affinity rate constant, which is obtained from the ratio of K_{d} to Kₐ. It can be measured by using surface plasma resonance method which is well known in the art.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “bispecific antibody” refers to a homogeneous antibody population involved in the highly specific recognition and binding of a two different antigenic determinants, or epitopes. The term “recombinant antibody”, as used herein, includes all antibodies that are prepared, expressed, created or isolated by recombinant means. In certain embodiments, however, such recombinant antibodies can be obtained by in vitro mutagenesis and thus the amino acid sequences of the V_{H} and V_{L} regions of the recombinant antibodies as described herein are sequences that may not naturally exist within the human antibody germline repertoire in vivo. The term “effector function” as used herein is a biochemical event that results from the interaction of an antibody Fc region with an Fc receptor or ligand. Effector functions include but are not limited to ADCC, ADCP, and CDC. The term also represents a physiological event such as circulating half-life of a drug or targeting of a drug to a particular cell or tissue type.

The term “ADCC” or “antibody dependent cell-mediated cytotoxicity” as used herein is the cell-mediated reaction wherein nonspecific cytotoxic cells that express FcγRs recognize bound antibody on a target cell and subsequently cause lysis of the target cell.

The term “ADCP” or “antibody dependent cell-mediated phagocytosis” as used herein is the cell-mediated reaction wherein nonspecific cytotoxic cells that express FcγRs recognize bound antibody on a target cell and subsequently cause phagocytosis of the target cell.

The term “effector cell” as used herein is a cell that expresses one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and γδT cells, and may be from any organism including but not limited to humans, mice, rats, rabbits, and monkeys.

The term “Fc” fragment, whose name reflects its ability to crystallize readily. The crystal structure of the human IgG Fc region has been determined (4). In human IgG molecules, the Fc region is generated by papain cleavage N-terminal to Cys 226. The Fc region is central to the effector functions of antibodies.

The term “Fc protein” as used herein refers to the portion of a single immunoglobulin heavy chain beginning in the hinge region just upstream of the papain cleavage site and ending at the C-terminus of the antibody. Accordingly, a complete Fc domain comprises at least a portion of a hinge (e.g., upper, middle, and/or lower hinge region) domain, a CH2 domain, and a CH3 domain.

The term “Fc variant containing protein” as used herein refers to any molecule that comprises an Fc variant of the current invention. Preferably, Fc variant containing protein includes antibody, fusion protein and peptibody. Antibody, fusion protein and peptibody as referred herein include any approved or under clinical trial or under pre-clinical trial antibody, fusion protein and peptibody.

The term “Fc variant containing molecule” as used herein refers to any molecule that comprises an Fc variant of the current invention. It can be fusion product where Fc variant of the current invention is linked or conjugated to a drug, wherein drug can be small peptide or receptor or toxin or any chemical molecule.

The term “Fc variant protein” or “Fc protein variant” or “Fc variant” as used herein is a Fc protein that differs from that of a wild type Fc protein by virtue of at least one amino acid modification. Fc variant may refer to the Fc variant itself, a composition comprising the Fc variant, or the amino acid sequence that encodes it. Preferably, the Fc variant has at least one amino acid modification compared to the parent protein, e.g. from about one to about ten amino acid modifications, and preferably from about one to about five amino acid modifications compared to the parent.

The term “EU position” as used herein refers to the amino acid position in the EU numbering convention for the Fc region described in reference 5.

The term “CH1 domain” as used herein refers to the first (most amino terminal) constant region domain of an immunoglobulin heavy chain that extends from about EU positions 118-215. The CH1 domain is adjacent to the V_{H} domain and amino terminal to the hinge region of an immunoglobulin heavy chain molecule, and does not form a part of the Fc region of an immunoglobulin heavy chain.

The term “hinge region” as used herein refers to the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (6). The Fc variant of the present invention can include all or a portion of a hinge region.

The term “CH2 domain” as used herein refers to the portion of a heavy chain immunoglobulin molecule that extends from about EU positions 231-340.

The term “CH3 domain” as used herein refers to the portion of a heavy chain immunoglobulin molecule that extends approximately 110 residues from N-terminus of the CH2 domain, e.g., from about position 341-446 (EU numbering system).

The term “Fc gamma receptor” or “FcγR” as used herein is meant any member of the family of proteins that bind the IgG antibody Fc region and is encoded by an FcγR gene. In humans, this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (7), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

The term “FcRn” or “neonatal Fc Receptor” as used herein is a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin.

The term “wild-type Fc” as used herein is an unmodified Fc polypeptide that is subsequently modified to generate a variant. The wild-type Fc may be a naturally occurring polypeptide, or recombinant version of a naturally occurring polypeptide. The wild-type Fc may refer to the unmodified Fc polypeptide itself, compositions that comprise the unmodified Fc polypeptide, or the amino acid sequence that encodes it.

The term “position” as used herein is a location in the sequence of a protein. Positions may be numbered sequentially or according to an established format, for example the EU index as in Kabat. For example, position 305 is a position in the human antibody IgG₁.

The terms “patient” and “subject” are used interchangeably and are used in their conventional sense to refer to a living organism suffering from or prone to a condition that can be prevented or treated by administration of a composition of the present invention, and includes both humans and non-human animals. Examples of subjects include, but are not limited to, humans, chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, adult, juvenile and new born individuals are of interest.

The term “residue” as used herein is a position in a protein and its associated amino acid identity. For example, Threonine 307 (also referred to as Thr307, also referred to as T307) is a residue in the human antibody IgG₁.

The term “immunoconjugate” or “conjugate” as used herein refers to a compound or a derivative thereof that is linked to a cell binding agent (i.e., antibody or Fc variant as described herein) and is defined by a generic formula: A-L-D, wherein A=cell binding agent or antibody or Fc variant of the present invention, L=linker and D=Drug (toxin or pharmaceutical drug). Immunoconjugates can also be defined by the generic formula in reverse order: A-L-D.

The term “linker” is any chemical moiety that is capable of linking a compound, usually a drug, such as a maytansinoid or auristatin, to a cell-binding agent such as an anti FcRn or a fragment thereof in a stable, covalent manner. Linkers can be susceptible to or be substantially resistant to acid-induced cleavage, light-induced cleavage, peptidase-induced cleavage, esterase-induced cleavage, and disulfide bond cleavage, at conditions under which the compound or the antibody remains active. Suitable linkers are well known in the art and include, for example, disulfide groups, thioether groups, acid labile groups, photolabile groups, peptidase labile groups and esterase labile groups. Linkers also include charged linkers, and hydrophilic forms thereof as described herein and know in the art.

The term an “antagonist” is one which inhibits or reduces biological activity of the antigen it binds, such as FcRn. In a certain embodiment antagonist substantially or completely inhibits the biological activity of the antigen such as FcRn. Desirably, the biological activity is reduced by 10%, 20%, 30%, 50%, 70%, 80%, 90%, 95%, or even 100%.

The term “treatment” or “therapeutics” as used herein, refers to any treatment of a disease in a mammal, particularly in a human. It includes: (a) preventing the disease from occurring in a subject which may be predisposed to the disease or at risk of acquiring the disease but has not yet been diagnosed as having it; (b) inhibiting the disease, i.e., arresting its development; and (c) relieving the disease, i.e., causing regression of the disease.

The term “wild type” or “WT” herein is an amino acid sequence or a nucleotide sequence that is found in nature, including allelic variations. A WT protein has an amino acid sequence or a nucleotide sequence that has not been intentionally modified.

The term “non-naturally encoded amino acid” refers to an amino acid that is not one of the common amino acids or pyrolysine, pyrroline-carboxy-lysine, or selenocysteine. Other terms that may be used synonymously with the term “non-naturally encoded amino acid” are “non-natural amino acid”, “unnatural amino acid”, “non-naturally-occurring amino acid” and variously hyphenated and non-hyphenated versions thereof. The term “non-naturally encoded amino acid” also includes, but is not limited to, amino acids that occur by modification (e.g. post-translational modifications) of a naturally encoded amino acid (including but not limited to, the 20 common amino acids or pyrrolysine, pyrroline-carboxy-lysine, and selenocysteine) but are not themselves naturally incorporated into a growing polypeptide chain by the translation complex. Examples of such non-naturally-occurring amino acids include, but are not limited to, N-acetylglucosaminyl-L-serine, N-acetylglucosaminyl-L-threonine and Ophosphotyrosine.

TABLE 1
Abbreviations of amino acid as used in the current application

| | Abbreviation | Abbreviation |
|---|---|---|
| Full Name | (3 Letter) | (1 Letter) |
| Alanine | Ala | A |
| Arginine | Arg | R |
| Asparagine | Asn | N |
| Aspartate | Asp | D |
| Cysteine | Cys | C |
| Glutamate | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

### Other Abbreviations Used in the Current Patent Application

%: percentage

° C.: degree Celsius

μg: microgram

μL: microliter

A: Adenine

ADCC: Antibody-dependent cellular cytotoxicity

C: Cytosine

CDC: Complement-dependent cytotoxicity

CFU: Colony forming unit

CHO: Chinese Hamster Ovary

DNA: Deoxyribonucleic acid

ELISA: Enzyme linked immunosorbent assay

EC₅₀: 50% of maximal effective concentration of any drug

EC₇₅: 75% of maximal effective concentration of any drug

Fc: Fragment crystallizable

FcGRT: Fc fragment of IgG receptor and transporter

FcRn: Neonatal Fc receptor

FcγRs: Fc gamma receptors

G: Guanine

h: Hour

H₂SO₄: Sulfuric acid

HRP: Horseradish peroxidase

IC: Immune-complex

IgG: Immunoglobulin G

IPTG: Isopropyl β-D-1-thiogalactopyranoside

ITP: Acute immune thrombocytopenia

IVIg: Intravenous immunoglobulin

Kₐ/k_{assoc}: Association constant

K_{d}/k_{dissoc}: Dissociation constant

K_{D}: Equilibrium dissociation constant

M: molar

mg: milligram

MgCl₂: Magnesium chloride

min: Minute

mL milliliter

mM: millimolar

MOI: Multiplicity of infection

NaCl: Sodium chloride

NaHCO₃: Sodium bicarbonate

ng: nanogram

nm: nanometer

OD: Optical density

OPD: o-phenylenediamine dihydrochloride

PBS: Phosphate buffered saline

PBST: Phosphate buffered saline with tween 0.05%

PEG: Polyethylene glycol

PFU: Plaque-forming unit

rFcRn: Recombinant neonatal Fc receptor

rhFcRn: Recombinant human neonatal Fc receptor

rpm: Round per minute

s: Second

SCIg: Subcutaneous immunoglobulin

SEQ/seq: Sequence

SPR: Surface plasmon resonance

T: Thymine

YT media: Yeast extract tryptone media

### EMBODIMENTS OF THE INVENTION

The disclosure of the present invention relates to novel Fc variant protein that can be used for therapeutic purposes.

In one embodiment, the Fc variant protein or Fc variant containing protein or Fc variant containing molecule of the present invention binds with high affinity to human FcRn relative to the wild-type Fc protein. In one of the embodiments, the Fc variant of the present invention has a K_{D} of 10⁻⁸ M or less, more preferably 10⁻¹⁰ M or less for FcRn. K_{D} value is a measurement of the binding affinity of the antibody towards its target antigen.

In one of the embodiments, the Fc variant according to the current invention has altered (increased or decreased) affinity for an Fc gamma receptor relative to the affinity of a wild-type IgG₁ Fc region for the said Fc gamma receptor. In certain embodiments, the Fc variant has increased affinity for FcγRIIIa (CD16a) relative to the affinity of a wild-type IgG₁ Fc region for FcγRIIIa (CD16a).

In one of the embodiments, the Fc variant of the present invention has a K_{D} of 10⁻⁷ M or less for FcγRIIIa (CD16a), including allotypes V158 and F158. In one of the embodiments, the Fc variant of the present invention inhibits immune-complex mediated FcγR activation as assessed by inhibition of ADCC. In one of the embodiments, the Fc variant of the present invention inhibits phagocytosis mediated by FcγRs. In one of the embodiments, the Fc variant of the present invention inhibits cytokine release such as IL-6 or IL-8 mediated by FcγRs.

In one embodiment, the amino acid sequence of Fc variant is of the IgG₁, IgG₂, IgG₃, IgG₄ or IgG₂/G₄ isotype, preferably the IgG₁ isotype.

In another embodiment, one or more Fc variant of the present invention has modified or reduced or no effector function. In one of the embodiments, the Fc variant or Fc variant containing protein of the present invention has reduced potential to cause the safety issue of ADCC and CDC. In one of the preferred embodiments, the Fc variant of the current invention has no CDC activity. In one of the preferred embodiments, the Fc variant of the current invention has reduced ADCC activity as compared to ADCC activity of wild-type Fc or ADCC activity of IVIg. In one of the embodiments, the Fc variant of the current invention has reduced or no ADCP activity. In one of the embodiments, the Fc variant of the present invention does not interfere with the binding property of FcRn to albumin.

In one of the embodiments, the Fc variant of the present invention cross-reacts with FcRn from species other than human.

In one of the embodiments, the Fc variant of the present invention has higher binding specificity towards human FcRn.

In one of the embodiments, Fc variant or Fc variant containing protein or Fc variant containing molecule of the present invention has an increased half-life in subject. In certain embodiments, polyethylene glycol or human serum albumin may link to Fc variant of the current invention to further increase the half-life of the said Fc variant. In an alternate embodiment, the Fc variant of the present invention may include mutations to increase its half-life in the subject. In another alternate embodiment, the Fc variant of the present invention can be expressed in multimer form to increase half-life by increasing the molecular size and affinity through higher avidity of the Fc variant. The term “multimer form” as used herein refers to a form of protein which includes more than one unit of protein, preferably herein Fc protein. For example, it can be dimer, trimer, tetramer, pentamer, hexamer, etc. For example, monomer Fc protein has two binding sites for FcRn as well as FcγRs. In the similar manner, Fc dimer according to the current invention has four binding sites for FcRn as well as FcγRs.

In another embodiment, the Fc variant of the present invention is able to bind to the monkey FcRn enabling ease of drug development by providing a relevant animal pharmacology and toxicology model.

In certain embodiments, the Fc variant comprises a variant Fc region that comprises an afucosylated N-linked glycan at EU position 297.

In one of the embodiments, the Fc variant comprises a variant Fc region that comprises higher sialylation as compared to wild-type IgG.

In one of the embodiments, the present invention provides a composition comprising Fc variant protein or Fc variant containing protein or Fc variant containing molecule and an acceptable carrier.

In another embodiment, the Fc variant protein or Fc variant containing protein of the present invention can be used for the treatment of disease such as infections, various cancers, auto immune disorders and the like.

In certain embodiments, the Fc variant or Fc variant containing protein or Fc variant containing molecule comprise an amino acid sequence selected from sequences as set forth in SEQ ID No. 1 to SEQ ID No. 347. Sequences with SEQ ID No.s from 1 to 347 are described herein in table 3.

In another embodiment, the Fc variant of the present invention can be used to make full-length antibody, where antibody has Fc variant of the present invention in place of conventional Fc region. Said antibody can be modified form of already approved or discovered antibody, where modified form of existing antibody has Fc variant of the current invention. It can be novel antibody which is developed for any target.

In a separate embodiment, the Fc variant of the present invention can be used to make a drug with increased half-life, where Fc variant of the present invention is either fused or linked or conjugated to drug of which original half-life in circulation is low.

In another embodiment, the Fc variant of the present invention can be used to increase the half-life of ADC and reducing its off-target toxicity by increasing its re-circulation through FcRn receptor.

In one of the embodiments, Fc variant of the present invention can be fused with any drug to improve its half-life and in-vivo stability.

In one of the embodiments, Fc variant of the present invention can be used to replace albumin in an albumin fusion drug, to improve the pharmacokinetics of the drug.

In one of the embodiments, mutations in the Fc variant of the present invention can be used to improve the affinity of full antibody towards FcRn at pH 6, to increase its circulating half-life by reducing catabolism.

In one of the embodiments, the Fc variant containing antibody molecule can be used as a scavenger to remove pathogenic proteins or toxins from the circulation such as TNF alpha, VEGF etc.

In one of the embodiments, the Fc variant containing antibody molecule or protein can be used as a trapper to catch desired proteins, peptides, carbohydrates or drugs from the environment and bring inside the FcRn expressing target cell.

In one of the embodiments, Fc variant of the present invention can be used to develop monomeric Fc fusion protein in fusion with any therapeutic peptide to improve its penetration in a tissue along with retaining its FcRn binding activity.

In one of the embodiments, Fc variant of the present invention can be fused with PEG to further improve its circulating half-life.

In one of the embodiments, Fc variant of the present invention can be used for delivering drugs via non-invasive route like pulmonary administration.

In one of the embodiments, Fc variant of the present invention blocks IgG binding site on FcRn and compete with endogenous IgG for FcRn leading to faster clearance of endogenous IgG.

In one of the embodiments, Fc variant of the present invention can improve the potency/efficacy of a drug independent of its pharmacokinetic half-life.

In one of the embodiments, Fc variant of the present invention in fusion with immunogen/antigen can improve the delivery of antigen to the FcRn expressing APCs leading to improved immune response.

In one of the embodiments, Fc variant of the present invention can be used to deliver nanoparticle laden drugs orally at various organs through intestinal epithelium.

In one of the embodiments, Fc variant of the present invention in fusion with a peptide/immunogen can be used for intranasal immunization as a general delivery route for subunit vaccines against many mucosal pathogens.

In one of the embodiments, Fc variant of the present invention in fusion with any broadly neutralizing antibody can be used for extending the half-life of such antibodies and also to enhance mucosal localization that confers immune protection against viral entry in the gastrointestinal or cervicovaginal tracts.

### DETAILED DESCRIPTION OF THE INVENTION

In one embodiment, the Fc variant protein or Fc variant containing protein or Fc variant containing molecule of the present invention binds with high affinity to human FcRn. The Fc variant of the current invention has lower K_{D} value than native Fc for binding to FcRn at pH 6.0. The Fc variant of the present invention has a K_{D} of 10⁻⁸ M or less, more preferably 10⁻¹⁰ M or less for FcRn at pH 6.0. K_{D} value is a measurement of the binding affinity of the antibody towards its target antigen.

Amino Acid Sequences of the Fc Variants

Fc variant of the present invention comprises amino acid substitution at CH2 domain or at CH3 domain or at CH2 and CH3 domain of the Fc protein. More preferably, the Fc variant of the present invention comprises amino acid substitution at one or more residue(s) selected from EU250, EU251, EU252, EU253, EU254, EU255, EU256, EU257, EU258, EU259, EU307, EU308, EU309, EU310, EU311, EU375, EU377, EU379, EU380, EU381, EU432, EU433, EU434 and EU435. Preferred substituting amino acids for the positions EU250, EU251, EU252, EU253, EU307, EU308, EU309, EU375, EU377, EU379, EU380, EU381, EU432, EU433, EU434, EU435, EU436 and EU437 are shown in table 2.

TABLE 2
Preferred substituting amino acid at specific EU position in Fc variant

| Residue | Substituting amino acid |
|---|---|
| EU250 | Alanine (A), Histidine (H), Valine (V), Lysine(K), Glutamine (Q) or Arginine |
| | (R) |
| EU251 | Glutamic acid (E), Glutamine (Q), Arginine (R), Tyrosine (Y), Cysteine (C), |
| | Valine (V), Histidine (H) or Alanine (A) |
| EU252 | Glutamine (Q), Tyrosine (Y), Histidine (H), Phenylalanine (F), Leucine (L) or |
| | Valine (V) |
| EU253 | Glutamine (Q), Arginine (R), Threonine (T), Asparagine (N), Proline (P), |
| | Serine (S) or Tyrosine (Y) |
| EU254 | Histidine (H) |
| EU255 | Glutamine (Q) |
| EU256 | Glutamine (Q) |
| EU257 | Glutamine (Q) |
| EU258 | Tyrosine (Y) or Tryptophan(W) |
| EU259 | Arginine (R) or Glycine (G) |
| EU307 | Asparagine (N), Tyrosine (Y) or Glutamine (Q) |
| EU308 | Proline (P), Aspartic acid (D), Threonine (T) or Serine (S) |
| EU309 | Tyrosine (Y), Serine (S), Threonine (T) or Glutamine (Q) |
| EU310 | Proline (P) or Valine (V) |
| EU311 | Histidine (H) or Asparagine (N) |
| EU375 | Arginine (R) |
| EU377 | Leucine (L) |
| EU379 | Isoleucine (I) |
| EU380 | Glutamine (Q) |
| EU381 | Glutamine (Q) |
| EU432 | Serine (S), Cysteine (C), Tryptophan (W), Phenylalanine (F), Alanine (A), |
| | Methionine (M), Glutamine (Q), Tyrosine (Y) or Proline (P) |
| EU433 | Arginine (R), Threonine (T), Glutamine (Q), Proline (P), Lysine (K), |
| | Phenylalanine (F), Serine (S), Asparagine (N) or Methionine (M) |
| EU434 | Tryptophan (W), Tyrosine (Y), Histidine (H), Phenylalanine (F), Aspartic |
| | acid (D), Glycine (G), Isoleucine (I), Leucine (L), Threonine (T) or |
| | Glutamine (Q) |
| EU435 | Glutamine (Q), Lysine (K), Proline (P), Asparagine (N) or Aspartic acid (D) |
| EU436 | Phenylalanine (F) |
| EU437 | Asparagine (N) |

In a preferred embodiment, the amino acid substitution of the Fc variant according to the present invention is selected from T250Q, T250R, T250A, T250H, T250V, T250K, L251H, L251A, L251E, L251Q, L251R, L251Y, L251C, L251V, M252L, M252V, M252Q, M252Y, M252H, M252F, I253P, I253S, I253Q, I253R, I253T, I253N, I253Y, S254H, R255Q, T256Q, P257Q, E258Y, E258W, V259R, V259G, T307N, T307Y, T307Q, V308P, V308D, V308T, V308S, L309Y, L309S, L309T, L309Q, H310P, H310V, Q311H, Q311N, S375R, I377L, V379I, E380Q, W381Q, L432S, L432C, L432Q, L432Y, L432P, L432W, L432F, L432A, L432M, H433R, H433T, H433Q, H433P, H433K, H433F, H433S, H433N, H433M, N434W, N434Y, N434H, N434F, N434D, N434G, N434I, N434L, N434T, N434Q, H435Q, H435K, H435P, H435N, H435D, Y436F, T437N and suitable combination thereof.

Fc variant of the present invention may comprise substitution at two or more positions in wild-type Fc, which is herein referred to as a “combination” of substitutions. The amino acid sequence of wild type Fc is mentioned as SEQ ID No. 348. Preferred combination of amino acid substitutions of Fc variant are shown in table 3. Amino acid substitutions that are present in combination are separated by the symbol “/”.

TABLE 3
Preferred combination of substituting amino
acids at specific EU position in Fc variant

| SEQ | |
|---|---|
| ID No. | Combination of amino acid substitutions |
| 1 | T250Q/L251H/M252L/I253P |
| 2 | T250R/L251A/M252V/I253S |
| 3 | T307N/V308P/L309Y |
| 4 | S375R/I377L |
| 5 | V379I/E380Q/W381Q |
| 6 | H433R/N434W |
| 7 | L432S/H433T/N434Y |
| 8 | H433R/N434Y |
| 9 | H433T/N434H/H435Q |
| 10 | H433Q/N434W |
| 11 | H433P/N434W/H435K |
| 12 | H433R/N434H/H435K |
| 13 | L432C/H433K/N434F |
| 14 | L432W/H433F/N434D/H435K |
| 15 | L432F/H433S/N434G |
| 16 | H433R/N434F |
| 17 | H433P/N434Y |
| 18 | L432A/H433S/N434F |
| 19 | H433P/N434W |
| 20 | L432M/H433P/N434F |
| 21 | L432A/H433K/N434F |
| 22 | T307N/V308P/L309Y/H433R/N434W |
| 23 | T307N/V308P/L309Y/L432S/H433T/N434Y |
| 24 | T307N/V308P/L309Y/H433R/N434Y |
| 25 | T250Q/L251H/M252L/I253P/H433T/N434H/H435Q |
| 26 | S375R/I377L/L432C/H433K/N434F |
| 27 | T307N/V308P/L309Y/V379I/E380Q/W381Q/H433R/N434W |
| 28 | T307Y/V308D |
| 29 | T250A/T307Y/V308D |
| 30 | T250H/L251E/M252Q/I253Q |
| 31 | T250A/L251Q/M252Y/I253R |
| 32 | T250A/L251R/M252H/I253T |
| 33 | T250V/L251Y/M252Q |
| 34 | T250K/L251C/M252Q/I253N |
| 35 | L251V/M252F/I253P |
| 36 | T256Q/P257Q/E258Y/V259R |
| 37 | E258W/V259G |
| 38 | I253Y/S254H/R255Q |
| 39 | T307Q/V308T/L309S |
| 40 | L309T/H310P/Q311H |
| 41 | V308S/L309Q/H310V/Q311N |
| 42 | L432M/H433P/N434I/H435P |
| 43 | L432Q/H433N/H435N |
| 44 | L432Y/H433R/N434L/H435P |
| 45 | N434T/H435D/Y436F/T437N |
| 46 | L432P/H433M/N434Q |
| 47 | V308S/L309Q/H310V/Q311N/L432Q/H433N/H435N |
| 48 | T250A/L251Q/M252Y/I253R/L432Y/H433R/N434L/H435P |
| 49 | E258W/V259G/N434T/H435D/Y436F/T437N |
| 50 | V379I/E380Q/W381Q/H433R/N434W |
| 51 | T250R/L251A/M252V/I253S/H433R/N434W |
| 52 | T250A/T307Y/V308D/H433R/N434W |
| 53 | T307Q/V308T/L309S/H433R/N434W |
| 54 | T256Q/P257Q/E258Y/V259R/H433R/N434W |
| 55 | T250H/L251E/M252Q/I253Q/H433R/N434W |
| 56 | L309T/H310P/Q311H/H433R/N434W |
| 57 | T250A/L251R/M252H/I253T/H433R/N434W |
| 58 | T250V/L251Y/M252Q/H433R/N434W |
| 59 | T250K/L251C/M252Q/I253N/H433R/N434W |
| 60 | I253Y/S254H/R255Q/H433R/N434W |
| 61 | L251V/M252F/I253P/H433R/N434W |
| 62 | V379I/E380Q/W381Q/L432S/H433T/N434Y |
| 63 | T250R/L251A/M252V/I253S/L432S/H433T/N434Y |
| 64 | T250A/T307Y/V308D/L432S/H433T/N434Y |
| 65 | T307Q/V308T/L309S/L432S/H433T/N434Y |
| 66 | T256Q/P257Q/E258Y/V259R/L432S/H433T/N434Y |
| 67 | T250H/L251E/M252Q/I253Q/L432S/H433T/N434Y |
| 68 | L309T/H310P/Q311H/L432S/H433T/N434Y |
| 69 | T250A/L251R/M252H/I253T/L432S/H433T/N434Y |
| 70 | T250V/L251Y/M252Q/L432S/H433T/N434Y |
| 71 | T250K/L251C/M252Q/I253N/L432S/H433T/N434Y |
| 72 | I253Y/S254H/R255Q/L432S/H433T/N434Y |
| 73 | L251V/M252F/I253P/L432S/H433T/N434Y |
| 74 | V379I/E380Q/W381Q/H433R/N434Y |
| 75 | T250R/L251A/M252V/I253S/H433R/N434Y |
| 76 | T250A/T307Y/V308D/H433R/N434Y |
| 77 | T307Q/V308T/L309S/H433R/N434Y |
| 78 | T256Q/P257Q/E258Y/V259R/H433R/N434Y |
| 79 | T250H/L251E/M252Q/I253Q/H433R/N434Y |
| 80 | L309T/H310P/Q311H/H433R/N434Y |
| 81 | T250A/L251R/M252H/I253T/H433R/N434Y |
| 82 | T250V/L251Y/M252Q/H433R/N434Y |
| 83 | T250K/L251C/M252Q/I253N/H433R/N434Y |
| 84 | I253Y/S254H/R255Q/H433R/N434Y |
| 85 | L251V/M252F/I253P/H433R/N434Y |
| 86 | T250Q/L251H/M252L/I253P/T307N/V308P/L309Y/H433T/ |
| | N434H/H435Q |
| 87 | T307N/V308P/L309Y/H433Q/N434W |
| 88 | T307N/V308P/L309Y/H433P/N434W/H435K |
| 89 | T307N/V308P/L309Y/H433R/N434H/H435K |
| 90 | T307N/V308P/L309Y/S375R/I377L/L432C/H433K/N434F |
| 91 | T307N/V308P/L309Y/L432W/H433F/N434D/H435K |
| 92 | T307N/V308P/L309Y/L432F/H433S/N434G |
| 93 | T307N/V308P/L309Y/V379I/E380Q/W381Q |
| 94 | T250R/L251A/M252V/I253S/T307N/V308P/L309Y |
| 95 | T307N/V308P/L309Y/H433R/N434F |
| 96 | T307N/V308P/L309Y/H433P/N434Y |
| 97 | T307N/V308P/L309Y/L432A/H433S/N434F |
| 98 | T307N/V308P/L309Y/H433P/N434W |
| 99 | T307N/V308P/L309Y/L432M/H433P/N434F |
| 100 | T307N/V308P/L309Y/L432A/H433K/N434F |
| 101 | T256Q/P257Q/E258Y/V259R/T307N/V308P/L309Y |
| 102 | T250H/L251E/M252Q/I253Q/T307N/V308P/L309Y |
| 103 | T307N/V308P/L309Y/L432M/H433P/N434I/H435P |
| 104 | T307N/V308P/L309Y/L432P/H433M/N434Q |
| 105 | T250A/L251Q/M252Y/I253R/T307N/V308P/L309Y/L432Y/ |
| | H433R/N434L/H435P |
| 106 | E258W/V259G/T307N/V308P/L309Y/N434T/H435D/Y436F/ |
| | T437N |
| 107 | T250A/L251R/M252H/I253T/T307N/V308P/L309Y |
| 108 | T250V/L251Y/M252Q/T307N/V308P/L309Y |
| 109 | T250K/L251C/M252Q/I253N/T307N/V308P/L309Y |
| 110 | I253Y/S254H/R255Q/T307N/V308P/L309Y |
| 111 | L251V/M252F/I253P/T307N/V308P/L309Y |
| 112 | T250Q/L251H/M252L/I253P/V379I/E380Q/W381Q/H433T/ |
| | N434H/H435Q |
| 113 | T250Q/L251H/M252L/I253P/T307Q/V308T/L309S/H433T/ |
| | N434H/H435Q |
| 114 | T250Q/L251H/M252L/I253P/T256Q/P257Q/E258Y/V259R/ |
| | H433T/N434H/H435Q |
| 115 | T250Q/L251H/M252L/I253P/L309T/H310P/Q311H/H433T/ |
| | N434H/H435Q |
| 116 | T250Q/L251H/M252L/I253P/1253Y/S254H/R255Q/H433T/ |
| | N434H/H435Q |
| 117 | V379I/E380Q/W381Q/H433Q/N434W |
| 118 | T250R/L251A/M252V/I253S/H433Q/N434W |
| 119 | T250A/T307Y/V308D/H433Q/N434W |
| 120 | T307Q/V308T/L309S/H433Q/N434W |
| 121 | T256Q/P257Q/E258Y/V259R/H433Q/N434W |
| 122 | T250H/L251E/M252Q/I253Q/H433Q/N434W |
| 123 | L309T/H310P/Q311H/H433Q/N434W |
| 124 | T250A/L251R/M252H/I253T/H433Q/N434W |
| 125 | T250V/L251Y/M252Q/H433Q/N434W |
| 126 | T250K/L251C/M252Q/I253N/H433Q/N434W |
| 127 | I253Y/S254H/R255Q/H433Q/N434W |
| 128 | L251V/M252F/I253P/H433Q/N434W |
| 129 | V379I/E380Q/W381Q/H433P/N434W/H435K |
| 130 | T250R/L251A/M252V/I253S/H433P/N434W/H435K |
| 131 | T250A/T307Y/V308D/H433P/N434W/H435K |
| 132 | T307Q/V308T/L309S/H433P/N434W/H435K |
| 133 | T256Q/P257Q/E258Y/V259R/H433P/N434W/H435K |
| 134 | T250H/L251E/M252Q/I253Q/H433P/N434W/H435K |
| 135 | L309T/H310P/Q311H/H433P/N434W/H435K |
| 136 | T250A/L251R/M252H/I253T/H433P/N434W/H435K |
| 137 | T250V/L251Y/M252Q/H433P/N434W/H435K |
| 138 | T250K/L251C/M252Q/I253N/H433P/N434W/H435K |
| 139 | I253Y/S254H/R255Q/H433P/N434W/H435K |
| 140 | L251V/M252F/I253P/H433P/N434W/H435K |
| 141 | V379I/E380Q/W381Q/H433R/N434H/H435K |
| 142 | T250R/L251A/M252V/I253S/H433R/N434H/H435K |
| 143 | T250A/T307Y/V308D/H433R/N434H/H435K |
| 144 | T307Q/V308T/L309S/H433R/N434H/H435K |
| 145 | T256Q/P257Q/E258Y/V259R/H433R/N434H/H435K |
| 146 | T250H/L251E/M252Q/I253Q/H433R/N434H/H435K |
| 147 | L309T/H310P/Q311H/H433R/N434H/H435K |
| 148 | T250A/L251R/M252H/I253T/H433R/N434H/H435K |
| 149 | T250V/L251Y/M252Q/H433R/N434H/H435K |
| 150 | T250K/L251C/M252Q/I253N/H433R/N434H/H435K |
| 151 | I253Y/S254H/R255Q/H433R/N434H/H435K |
| 152 | L251V/M252F/I253P/H433R/N434H/H435K |
| 153 | S375R/I377L/V379I/E380Q/W381Q/L432C/H433K/N434F |
| 154 | T250R/L251A/M252V/I253S/S375R/I377L/L432C/H433K/ |
| | N434F |
| 155 | T250A/T307Y/V308D/S375R/I377L/L432C/H433K/N434F |
| 156 | T307Q/V308T/L309S/S375R/I377L/L432C/H433K/N434F |
| 157 | T256Q/P257Q/E258Y/V259R/S375R/I377L/L432C/H433K/ |
| | N434F |
| 158 | T250H/L251E/M252Q/I253Q/S375R/I377L/L432C/H433K/ |
| | N434F |
| 159 | L309T/H310P/Q311H/S375R/I377L/L432C/H433K/N434F |
| 160 | T250A/L251R/M252H/I253T/S375R/I377L/L432C/H433K/ |
| | N434F |
| 161 | T250V/L251Y/M252Q/S375R/I377L/L432C/H433K/N434F |
| 162 | V379I/E380Q/W381Q/L432W/H433F/N434D/H435K |
| 163 | T250R/L251A/M252V/I253S/L432W/H433F/N434D/H435K |
| 164 | T250A/T307Y/V308D/L432W/H433F/N434D/H435K |
| 165 | T307Q/V308T/L309S/L432W/H433F/N434D/H435K |
| 166 | T256Q/P257Q/E258Y/V259R/L432W/H433F/N434D/H435K |
| 167 | T250H/L251E/M252Q/I253Q/L432W/H433F/N434D/H435K |
| 168 | L309T/H310P/Q311H/L432W/H433F/N434D/H435K |
| 169 | T250A/L251R/M252H/I253T/L432W/H433F/N434D/H435K |
| 170 | T250V/L251Y/M252Q/L432W/H433F/N434D/H435K |
| 171 | T250K/L251C/M252Q/I253N/L432W/H433F/N434D/H435K |
| 172 | I253Y/S254H/R255Q/L432W/H433F/N434D/H435K |
| 173 | L251V/M252F/I253P/L432W/H433F/N434D/H435K |
| 174 | V379I/E380Q/W381Q/L432F/H433S/N434G |
| 175 | T250R/L251A/M252V/I253S/L432F/H433S/N434G |
| 176 | T250A/T307Y/V308D/L432F/H433S/N434G |
| 177 | T307Q/V308T/L309S/L432F/H433S/N434G |
| 178 | T256Q/P257Q/E258Y/V259R/L432F/H433S/N434G |
| 179 | T250H/L251E/M252Q/I253Q/L432F/H433S/N434G |
| 180 | L309T/H310P/Q311H/L432F/H433S/N434G |
| 181 | T250A/L251R/M252H/I253T/L432F/H433S/N434G |
| 182 | T250V/L251Y/M252Q/L432F/H433S/N434G |
| 183 | I253Y/S254H/R255Q/L432F/H433S/N434G |
| 184 | L251V/M252F/I253P/L432F/H433S/N434G |
| 185 | T250R/L251A/M252V/I253S/V379I/E380Q/W381Q |
| 186 | V379I/E380Q/W381Q/H433R/N434F |
| 187 | V379I/E380Q/W381Q/H433P/N434Y |
| 188 | V379I/E380Q/W381Q/L432A/H433S/N434F |
| 189 | V379I/E380Q/W381Q/H433P/N434W |
| 190 | V379I/E380Q/W381Q/L432M/H433P/N434F |
| 191 | V379I/E380Q/W381Q/L432A/H433K/N434F |
| 192 | T250A/T307Y/V308D/V379I/E380Q/W381Q |
| 193 | T307Q/V308T/L309S/V379I/E380Q/W381Q |
| 194 | T256Q/P257Q/E258Y/V259R/V379I/E380Q/W381Q |
| 195 | T250H/L251E/M252Q/I253Q/V379I/E380Q/W381Q |
| 196 | L309T/H310P/Q311H/V379I/E380Q/W381Q |
| 197 | V379I/E380Q/W381Q/L432M/H433P/N434I/H435P |
| 198 | V379I/E380Q/W381Q/L432P/H433M/N434Q |
| 199 | V308S/L309Q/H310V/Q311N/V379I/E380Q/W381Q/L432Q/ |
| | H433N/H435N |
| 200 | T250A/L251Q/M252Y/I253R/V379I/E380Q/W381Q/L432Y/ |
| | H433R/N434L/H435P |
| 201 | E258W/V259G/V379I/E380Q/W381Q/N434T/H435D/Y436F/ |
| | T437N |
| 202 | T250A/L251R/M252H/I253T/V379I/E380Q/W381Q |
| 203 | T250V/L251Y/M252Q/V379I/E380Q/W381Q |
| 204 | T250K/L251C/M252Q/I253N/V379I/E380Q/W381Q |
| 205 | I253Y/S254H/R255Q/V379I/E380Q/W381Q |
| 206 | L251V/M252F/I253P/V379I/E380Q/W381Q |
| 207 | T250R/L251A/M252V/I253S/H433R/N434F |
| 208 | T250R/L251A/M252V/I253S/H433P/N434Y |
| 209 | T250R/L251A/M252V/I253S/L432A/H433S/N434F |
| 210 | T250R/L251A/M252V/I253S/H433P/N434W |
| 211 | T250R/L251A/M252V/I253S/L432M/H433P/N434F |
| 212 | T250R/L251A/M252V/I253S/L432A/H433K/N434F |
| 213 | T250R/L251A/M252V/I253S/T307Q/V308T/L309S/H433T/ |
| | N434H/H435Q |
| 214 | T250R/L251A/M252V/I253S/T256Q/P257Q/E258Y/V259R/ |
| | H433T/N434H/H435Q |
| 215 | T250R/L251A/M252V/I253S/L309T/H310P/Q311H/H433T/ |
| | N434H/H435Q |
| 216 | T250R/L251A/M252V/I253S/L432M/H433P/N434I/H435P |
| 217 | T250R/L251A/M252V/I253S/L432P/H433M/N434Q |
| 218 | T250R/L251A/M252V/I253S/V308S/L309Q/H310V/Q311N/ |
| | L432Q/H433N/H435N |
| 219 | T250R/L251A/M252V/I253S/E258W/V259G/N434T/H435D/ |
| | Y436F/T437N |
| 220 | T250R/L251A/M252V/I253S/1253Y/S254H/R255Q |
| 221 | T250A/T307Y/V308D/H433R/N434F |
| 222 | T307Q/V308T/L309S/H433R/N434F |
| 223 | T256Q/P257Q/E258Y/V259R/H433R/N434F |
| 224 | T250H/L251E/M252Q/I253Q/H433R/N434F |
| 225 | L309T/H310P/Q311H/H433R/N434F |
| 226 | T250A/L251R/M252H/I253T/H433R/N434F |
| 227 | T250V/L251Y/M252Q/H433R/N434F |
| 228 | T250K/L251C/M252Q/I253N/H433R/N434F |
| 229 | I253Y/S254H/R255Q/H433R/N434F |
| 230 | L251V/M252F/I253P/H433R/N434F |
| 231 | T250A/T307Y/V308D/H433P/N434Y |
| 232 | T307Q/V308T/L309S/H433P/N434Y |
| 233 | T256Q/P257Q/E258Y/V259R/H433P/N434Y |
| 234 | T250H/L251E/M252Q/I253Q/H433P/N434Y |
| 235 | L309T/H310P/Q311H/H433P/N434Y |
| 236 | T250A/L251R/M252H/I253T/H433P/N434Y |
| 237 | T250V/L251Y/M252Q/H433P/N434Y |
| 238 | T250K/L251C/M252Q/I253N/H433P/N434Y |
| 239 | I253Y/S254H/R255Q/H433P/N434Y |
| 240 | L251V/M252F/I253P/H433P/N434Y |
| 241 | T250A/T307Y/V308D/L432A/H433S/N434F |
| 242 | T307Q/V308T/L309S/L432A/H433S/N434F |
| 243 | T256Q/P257Q/E258Y/V259R/L432A/H433S/N434F |
| 244 | T250H/L251E/M252Q/I253Q/L432A/H433S/N434F |
| 245 | L309T/H310P/Q311H/L432A/H433S/N434F |
| 246 | T250A/L251R/M252H/I253T/L432A/H433S/N434F |
| 247 | T250V/L251Y/M252Q/L432A/H433S/N434F |
| 248 | T250K/L251C/M252Q/I253N/L432A/H433S/N434F |
| 249 | I253Y/S254H/R255Q/L432A/H433S/N434F |
| 250 | L251V/M252F/I253P/L432A/H433S/N434F |
| 251 | T250A/T307Y/V308D/H433P/N434W |
| 252 | T307Q/V308T/L309S/H433P/N434W |
| 253 | T256Q/P257Q/E258Y/V259R/H433P/N434W |
| 254 | T250H/L251E/M252Q/I253Q/H433P/N434W |
| 255 | L309T/H310P/Q311H/H433P/N434W |
| 256 | T250A/L251R/M252H/I253T/H433P/N434W |
| 257 | T250V/L251Y/M252Q/H433P/N434W |
| 258 | T250K/L251C/M252Q/I253N/H433P/N434W |
| 259 | I253Y/S254H/R255Q/H433P/N434W |
| 260 | L251V/M252F/I253P/H433P/N434W |
| 261 | T250A/T307Y/V308D/L432M/H433P/N434F |
| 262 | T307Q/V308T/L309S/L432M/H433P/N434F |
| 263 | T256Q/P257Q/E258Y/V259R/L432M/H433P/N434F |
| 264 | T250H/L251E/M252Q/I253Q/L432M/H433P/N434F |
| 265 | L309T/H310P/Q311H/L432M/H433P/N434F |
| 266 | T250A/L251R/M252H/I253T/L432M/H433P/N434F |
| 267 | T250V/L251Y/M252Q/L432M/H433P/N434F |
| 268 | T250K/L251C/M252Q/I253N/L432M/H433P/N434F |
| 269 | I253Y/S254H/R255Q/L432M/H433P/N434F |
| 270 | L251V/M252F/I253P/L432M/H433P/N434F |
| 271 | T250A/T307Y/V308D/L432A/H433K/N434F |
| 272 | T307Q/V308T/L309S/L432A/H433K/N434F |
| 273 | T256Q/P257Q/E258Y/V259R/L432A/H433K/N434F |
| 274 | T250H/L251E/M252Q/I253Q/L432A/H433K/N434F |
| 275 | L309T/H310P/Q311H/L432A/H433K/N434F |
| 276 | T250A/L251R/M252H/I253T/L432A/H433K/N434F |
| 277 | T250V/L251Y/M252Q/L432A/H433K/N434F |
| 278 | T250K/L251C/M252Q/I253N/L432A/H433K/N434F |
| 279 | I253Y/S254H/R255Q/L432A/H433K/N434F |
| 280 | L251V/M252F/I253P/L432A/H433K/N434F |
| 281 | T250A/T256Q/P257Q/E258Y/V259R/T307Y/V308D |
| 282 | T250A/T307Y/V308D/L432M/H433P/N434I/H435P |
| 283 | T250A/T307Y/V308D/L432P/H433M/N434Q |
| 284 | T250A/E258W/V259G/T307Y/V308D/N434T/H435D/Y436F/ |
| | T437N |
| 285 | T250A/I253Y/S254H/R255Q/T307Y/V308D |
| 286 | T256Q/P257Q/E258Y/V259R/T307Q/V308T/L309S |
| 287 | T250H/L251E/M252Q/I253Q/T307Q/V308T/L309S |
| 288 | T307Q/V308T/L309S/L432M/H433P/N434I/H435P |
| 289 | T307Q/V308T/L309S/L432P/H433M/N434Q |
| 290 | T250A/L251Q/M252Y/I253R/T307Q/V308T/L309S/L432Y/ |
| | H433R/N434L/H435P |
| 291 | E258W/V259G/T307Q/V308T/L309S/N434T/H435D/Y436F/ |
| | T437N |
| 292 | T250A/L251R/M252H/I253T/T307Q/V308T/L309S |
| 293 | T250V/L251Y/M252Q/T307Q/V308T/L309S |
| 294 | T250K/L251C/M252Q/I253N/T307Q/V308T/L309S |
| 295 | I253Y/S254H/R255Q/T307Q/V308T/L309S |
| 296 | L251V/M252F/I253P/T307Q/V308T/L309S |
| 297 | T250H/L251E/M252Q/I253Q/T256Q/P257Q/E258Y/V259R |
| 298 | T256Q/P257Q/E258Y/V259R/L309T/H310P/Q311H |
| 299 | T256Q/P257Q/E258Y/V259R/L432M/H433P/N434I/H435P |
| 300 | T256Q/P257Q/E258Y/V259R/L432P/H433M/N434Q |
| 301 | T256Q/P257Q/E258Y/V259R/V308S/L309Q/H310V/Q311N/ |
| | L432Q/H433N/H435N |
| 302 | T250A/L251Q/M252Y/I253R/T256Q/P257Q/E258Y/V259R/ |
| | L432Y/H433R/N434L/H435P |
| 303 | T250A/L251R/M252H/I253T/T256Q/P257Q/E258Y/V259R |
| 304 | T250V/L251Y/M252Q/T256Q/P257Q/E258Y/V259R |
| 305 | T250K/L251C/M252Q/I253N/T256Q/P257Q/E258Y/V259R |
| 306 | L251V/M252F/I253P/T256Q/P257Q/E258Y/V259R |
| 307 | T250H/L251E/M252Q/I253Q/L309T/H310P/Q311H |
| 308 | T250H/L251E/M252Q/I253Q/L432M/H433P/N434I/H435P |
| 309 | T250H/L251E/M252Q/I253Q/L432P/H433M/N434Q |
| 310 | T250H/L251E/M252Q/I253Q/V308S/L309Q/H310V/Q311N/ |
| | L432Q/H433N/H435N |
| 311 | T250H/L251E/M252Q/I253Q/E258W/V259G/N434T/H435D/ |
| | Y436F/T437N |
| 312 | T250H/L251E/M252Q/I253Q/T307Q/V308T/L309S |
| 313 | T250H/L251E/M252Q/I253Q/1253Y/S254H/R255Q |
| 314 | L309T/H310P/Q311H/L432M/H433P/N434I/H435P |
| 315 | L309T/H310P/Q311H/L432P/H433M/N434Q |
| 316 | T250A/L251Q/M252Y/I253R/L309T/H310P/Q311H/L432Y/ |
| | H433R/N434L/H435P |
| 317 | E258W/V259G/L309T/H310P/Q311H/N434T/H435D/Y436F/ |
| | T437N |
| 318 | T250A/L251R/M252H/I253T/L309T/H310P/Q311H |
| 319 | T250V/L251Y/M252Q/L309T/H310P/Q311H |
| 320 | T256Q/P257Q/E258Y/V259R/L309T/H310P/Q311H |
| 321 | T250K/L251C/M252Q/I253N/L309T/H310P/Q311H |
| 322 | I253Y/S254H/R255Q/L309T/H310P/Q311H |
| 323 | L251V/M252F/I253P/L309T/H310P/Q311H |
| 324 | T250A/L251R/M252H/I253T/L432M/H433P/N434I/H435P |
| 325 | T250V/L251Y/M252Q/L432M/H433P/N434I/H435P |
| 326 | T250K/L251C/M252Q/I253N/L432M/H433P/N434I/H435P |
| 327 | I253Y/S254H/R255Q/L432M/H433P/N434I/H435P |
| 328 | L251V/M252F/I253P/L432M/H433P/N434I/H435P |
| 329 | T250A/L251R/M252H/I253T/L432P/H433M/N434Q |
| 330 | T250V/L251Y/M252Q/L432P/H433M/N434Q |
| 331 | T250K/L251C/M252Q/I253N/L432P/H433M/N434Q |
| 332 | I253Y/S254H/R255Q/L432P/H433M/N434Q |
| 333 | L251V/M252F/I253P/L432P/H433M/N434Q |
| 334 | T250A/L251R/M252H/I253T/V308S/L309Q/H310V/Q311N/ |
| | L432Q/H433N/H435N |
| 335 | T250V/L251Y/M252Q/V308S/L309Q/H310V/Q311N/L432Q/ |
| | H433N/H435N |
| 336 | T250K/L251C/M252Q/I253N/V308S/L309Q/H310V/Q311N/ |
| | L432Q/H433N/H435N |
| 337 | I253Y/S254H/R255Q/V308S/L309Q/H310V/Q311N/L432Q/ |
| | H433N/H435N |
| 338 | L251V/M252F/I253P/V308S/L309Q/H310V/Q311N/L432Q/ |
| | H433N/H435N |
| 339 | T250A/L251Q/M252Y/I253R/I253Y/S254H/R255Q/L432Y/ |
| | H433R/N434L/H435P |
| 340 | T250A/L251R/M252H/I253T/E258W/V259G/N434T/H435D/ |
| | Y436F/T437N |
| 341 | T250V/L251Y/M252Q/E258W/V259G/N434T/H435D/Y436F/ |
| | T437N |
| 342 | T250K/L251C/M252Q/I253N/E258W/V259G/N434T/H435D/ |
| | Y436F/T437N |
| 343 | L251V/M252F/I253P/E258W/V259G/N434T/H435D/Y436F/ |
| | T437N |
| 344 | T250A/L251R/M252H/I253T/1253Y/S254H/R255Q |
| 345 | T250V/L251Y/M252Q/I253Y/S254H/R255Q |
| 346 | T250K/L251C/M252Q/I253N/I253Y/S254H/R255Q |
| 347 | L251V/M252F/I253P/1253Y/S254H/R255Q |

In a preferred embodiment, the combination of substituting amino acids at specific EU position in Fc variant according to the current invention is selected from the sequences as set forth in SEQ ID NO. 1, SEQ ID NO. 2, SEQ ID NO. 3, SEQ ID NO. 4, SEQ ID NO. 5, SEQ ID NO. 7, SEQ ID NO. 9, SEQ ID NO. 11, SEQ ID NO. 14, SEQ ID NO. 15, SEQ ID NO. 18, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 29, SEQ ID NO. 30, SEQ ID NO. 31, SEQ ID NO. 32, SEQ ID NO. 34, SEQ ID NO. 35, SEQ ID NO. 36, SEQ ID NO. 38, SEQ ID NO. 39, SEQ ID NO. 40, SEQ ID NO. 41, SEQ ID NO. 42, SEQ ID NO. 43, SEQ ID NO. 44, SEQ ID NO. 45, SEQ ID NO. 46, SEQ ID NO. 47, SEQ ID NO. 48, SEQ ID NO. 49, SEQ ID NO. 50, SEQ ID NO. 51, SEQ ID NO. 52, SEQ ID NO. 53, SEQ ID NO. 54, SEQ ID NO. 55, SEQ ID NO. 56, SEQ ID NO. 57, SEQ ID NO. 58, SEQ ID NO. 59, SEQ ID NO. 60, SEQ ID NO. 61, SEQ ID NO. 62, SEQ ID NO. 63, SEQ ID NO. 64, SEQ ID NO. 65, SEQ ID NO. 66, SEQ ID NO. 67, SEQ ID NO. 68, SEQ ID NO. 69, SEQ ID NO. 70, SEQ ID NO. 71, SEQ ID NO. 72, SEQ ID NO. 73, SEQ ID NO. 74, SEQ ID NO. 75, SEQ ID NO. 76, SEQ ID NO. 77, SEQ ID NO. 78, SEQ ID NO. 79, SEQ ID NO. 80, SEQ ID NO. 81, SEQ ID NO. 82, SEQ ID NO. 83, SEQ ID NO. 84, SEQ ID NO. 85, SEQ ID NO. 86, SEQ ID NO. 87, SEQ ID NO. 88, SEQ ID NO. 89, SEQ ID NO. 90, SEQ ID NO. 91, SEQ ID NO. 92, SEQ ID NO. 93, SEQ ID NO. 94, SEQ ID NO. 95, SEQ ID NO. 96, SEQ ID NO. 97, SEQ ID NO. 98, SEQ ID NO. 99, SEQ ID NO. 100, SEQ ID NO. 101, SEQ ID NO. 102, SEQ ID NO. 103, SEQ ID NO. 104, SEQ ID NO. 105, SEQ ID NO. 106, SEQ ID NO. 107, SEQ ID NO. 108, SEQ ID NO. 109, SEQ ID NO. 110, SEQ ID NO. 111, SEQ ID NO. 112, SEQ ID NO. 113, SEQ ID NO. 114, SEQ ID NO. 115, SEQ ID NO. 116, SEQ ID NO. 117, SEQ ID NO. 118, SEQ ID NO. 119, SEQ ID NO. 120, SEQ ID NO. 121, SEQ ID NO. 122, SEQ ID NO. 123, SEQ ID NO. 124, SEQ ID NO. 125, SEQ ID NO. 126, SEQ ID NO. 127, SEQ ID NO. 128, SEQ ID NO. 129, SEQ ID NO. 130, SEQ ID NO. 131, SEQ ID NO. 132, SEQ ID NO. 133, SEQ ID NO. 134, SEQ ID NO. 135, SEQ ID NO. 136, SEQ ID NO. 137, SEQ ID NO. 138, SEQ ID NO. 139, SEQ ID NO. 140, SEQ ID NO. 141, SEQ ID NO. 142, SEQ ID NO. 143, SEQ ID NO. 144, SEQ ID NO. 145, SEQ ID NO. 146, SEQ ID NO. 147, SEQ ID NO. 148, SEQ ID NO. 149, SEQ ID NO. 150, SEQ ID NO. 151, SEQ ID NO. 152, SEQ ID NO. 153, SEQ ID NO. 154, SEQ ID NO. 155, SEQ ID NO. 156, SEQ ID NO. 157, SEQ ID NO. 158, SEQ ID NO. 159, SEQ ID NO. 160, SEQ ID NO. 161, SEQ ID NO. 162, SEQ ID NO. 163, SEQ ID NO. 164, SEQ ID NO. 165, SEQ ID NO. 166, SEQ ID NO. 167, SEQ ID NO. 168, SEQ ID NO. 169, SEQ ID NO. 170, SEQ ID NO. 171, SEQ ID NO. 172, SEQ ID NO. 173, SEQ ID NO. 174, SEQ ID NO. 175, SEQ ID NO. 176, SEQ ID NO. 177, SEQ ID NO. 178, SEQ ID NO. 179, SEQ ID NO. 180, SEQ ID NO. 181, SEQ ID NO. 182, SEQ ID NO. 183, SEQ ID NO. 184, SEQ ID NO. 185, SEQ ID NO. 186, SEQ ID NO. 187, SEQ ID NO. 188, SEQ ID NO. 189, SEQ ID NO. 190, SEQ ID NO. 191, SEQ ID NO. 192, SEQ ID NO. 193, SEQ ID NO. 194, SEQ ID NO. 195, SEQ ID NO. 196, SEQ ID NO. 197, SEQ ID NO. 198, SEQ ID NO. 199, SEQ ID NO. 200, SEQ ID NO. 201, SEQ ID NO. 202, SEQ ID NO. 203, SEQ ID NO. 204, SEQ ID NO. 205, SEQ ID NO. 206, SEQ ID NO. 207, SEQ ID NO. 208, SEQ ID NO. 209, SEQ ID NO. 210, SEQ ID NO. 211, SEQ ID NO. 212, SEQ ID NO. 213, SEQ ID NO. 214, SEQ ID NO. 215, SEQ ID NO. 216, SEQ ID NO. 217, SEQ ID NO. 218, SEQ ID NO. 219, SEQ ID NO. 220, SEQ ID NO. 221, SEQ ID NO. 222, SEQ ID NO. 223, SEQ ID NO. 224, SEQ ID NO. 225, SEQ ID NO. 226, SEQ ID NO. 227, SEQ ID NO. 228, SEQ ID NO. 229, SEQ ID NO. 230, SEQ ID NO. 231, SEQ ID NO. 232, SEQ ID NO. 233, SEQ ID NO. 234, SEQ ID NO. 235, SEQ ID NO. 236, SEQ ID NO. 237, SEQ ID NO. 238, SEQ ID NO. 239, SEQ ID NO. 240, SEQ ID NO. 241, SEQ ID NO. 242, SEQ ID NO. 243, SEQ ID NO. 244, SEQ ID NO. 245, SEQ ID NO. 246, SEQ ID NO. 247, SEQ ID NO. 248, SEQ ID NO. 249, SEQ ID NO. 250, SEQ ID NO. 251, SEQ ID NO. 252, SEQ ID NO. 253, SEQ ID NO. 254, SEQ ID NO. 255, SEQ ID NO. 256, SEQ ID NO. 257, SEQ ID NO. 258, SEQ ID NO. 259, SEQ ID NO. 260, SEQ ID NO. 261, SEQ ID NO. 262, SEQ ID NO. 263, SEQ ID NO. 264, SEQ ID NO. 265, SEQ ID NO. 266, SEQ ID NO. 267, SEQ ID NO. 268, SEQ ID NO. 269, SEQ ID NO. 270, SEQ ID NO. 271, SEQ ID NO. 272, SEQ ID NO. 273, SEQ ID NO. 274, SEQ ID NO. 275, SEQ ID NO. 276, SEQ ID NO. 277, SEQ ID NO. 278, SEQ ID NO. 279, SEQ ID NO. 280, SEQ ID NO. 281, SEQ ID NO. 282, SEQ ID NO. 283, SEQ ID NO. 284, SEQ ID NO. 285, SEQ ID NO. 286, SEQ ID NO. 287, SEQ ID NO. 288, SEQ ID NO. 289, SEQ ID NO. 290, SEQ ID NO. 291, SEQ ID NO. 292, SEQ ID NO. 293, SEQ ID NO. 294, SEQ ID NO. 295, SEQ ID NO. 296, SEQ ID NO. 297, SEQ ID NO. 298, SEQ ID NO. 299, SEQ ID NO. 300, SEQ ID NO. 301, SEQ ID NO. 302, SEQ ID NO. 303, SEQ ID NO. 304, SEQ ID NO. 305, SEQ ID NO. 306, SEQ ID NO. 307, SEQ ID NO. 308, SEQ ID NO. 309, SEQ ID NO. 310, SEQ ID NO. 311, SEQ ID NO. 312, SEQ ID NO. 313, SEQ ID NO. 314, SEQ ID NO. 315, SEQ ID NO. 316, SEQ ID NO. 317, SEQ ID NO. 318, SEQ ID NO. 319, SEQ ID NO. 320, SEQ ID NO. 321, SEQ ID NO. 322, SEQ ID NO. 323, SEQ ID NO. 324, SEQ ID NO. 325, SEQ ID NO. 326, SEQ ID NO. 327, SEQ ID NO. 328, SEQ ID NO. 329, SEQ ID NO. 330, SEQ ID NO. 331, SEQ ID NO. 332, SEQ ID NO. 333, SEQ ID NO. 334, SEQ ID NO. 335, SEQ ID NO. 336, SEQ ID NO. 337, SEQ ID NO. 338, SEQ ID NO. 339, SEQ ID NO. 340, SEQ ID NO. 341, SEQ ID NO. 342, SEQ ID NO. 343, SEQ ID NO. 344, SEQ ID NO. 345, SEQ ID NO. 346 and SEQ ID NO. 347.

In one of the preferred embodiment, the combination of substituting amino acids at specific EU position in Fc variant according to the current invention is selected from the sequences as set forth in SEQ ID NO. 1, SEQ ID NO. 2, SEQ ID NO. 3, SEQ ID NO. 4, SEQ ID NO. 5, SEQ ID NO. 7, SEQ ID NO. 9, SEQ ID NO. 11, SEQ ID NO. 14, SEQ ID NO. 15, SEQ ID NO. 18, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 29, SEQ ID NO. 30, SEQ ID NO. 31, SEQ ID NO. 32, SEQ ID NO. 34, SEQ ID NO. 35, SEQ ID NO. 36, SEQ ID NO. 38, SEQ ID NO. 39, SEQ ID NO. 40, SEQ ID NO. 41, SEQ ID NO. 42, SEQ ID NO. 43, SEQ ID NO. 44, SEQ ID NO. 45, SEQ ID NO. 46, SEQ ID NO. 47, SEQ ID NO. 48 and SEQ ID NO. 49.

In one of the preferred embodiments, the combination of substituting amino acids at specific EU position in Fc variant according to the current invention is as set forth in SEQ ID NO. 3, SEQ ID NO. 5, SEQ ID NO. 10, SEQ ID NO. 20 or SEQ ID NO. 22.

In a more preferred embodiment, the amino acid sequence of Fc variant of the current invention is selected from SEQ ID NO. 22a, SEQ ID NO. 22b, SEQ ID NO. 22c, SEQ ID NO. 22d, SEQ ID NO. 22e and SEQ ID NO. 22f and SEQ ID NO. 22g.

The residues of Fc variant, their respective amino acid substitution and combinations of amino acid substitutions of specific residues, as described herein can be present in Fc protein of IgG₁, IgG₂, IgG₃, IgG₄ or IgG₂/G₄ isotype, preferably the IgG₁ isotype. Fc variant construct(s) of IgG2, IgG4 or IgG₂/G₄ isotype may contain single amino acid substitution (i.e., S228P) in the hinge region of Fc variant to reduce the disruption of disulphide bond between two Fc chains. (8) The Fc variant prepared according to the current invention may optionally be modified to modify functionality, e.g., to eliminate residual effector functions such as ADCC and CDC activity. Preferably, the Fc variant of the current invention has no CDC activity. Preferably, the Fc variants of the current invention has reduced ADCC activity as compared to ADCC activity of wild-type Fc or ADCC activity of IVIg. The Fc variant according to the current invention has altered (increased or decreased) affinity for an Fc receptor relative to the affinity of a wild-type IgG₁ Fc region for the said Fc gamma receptor. In one of the embodiments, the Fc variant has increased affinity for FcγRIIIa (CD16a), FcγRIIa (CD32a) and FcγRI (CD64) relative to the affinity of a wild-type IgG₁ Fc region for said FcγRIIIa (CD16a), FcγRIIa (CD32a) and FcγRI (CD64). In one of the embodiments, the Fc variant of the present invention has a K_{D} of 10⁻⁷ M or less for FcγRIIIa (CD16a), including allotypes V158 and F158. K_{D} value is a measurement of the binding affinity of the antibody towards its target antigen. In one of the embodiments, the Fc variant of the present invention inhibits immune complex mediated FcγR activation as assessed by inhibition of ADCC. In one of the embodiments, the Fc variant of the present invention inhibits phagocytosis mediated by FcγRs. In one of the embodiments, the Fc variant of the present invention inhibits cytokine release such as IL-6 or IL-8 mediated by FcγRs. Current invention provides Fc variant which can bind to FcRn as well as FcγRs with high affinity. Thus, Fc variant of the present invention has the combined effects of the multiple mechanisms of action of a single drug, such as FcRn blockade and inhibition of FcγR activation and thus it results into robust efficacy of the Fc variant of the current invention.

The combined effects has been illustrated herein examples with one of the non-limiting Fc variants of the current invention. The said Fc variant has substitutions of SEQ ID No. 22 as described in table 3. Amino acid sequence of non-limiting Fc variant of the current invention is as provided in below table 4. A particular variant SEQ ID NO. 22b of SEQ ID NO.22 was used in different experiments given herein examples.

TABLE 4
Amino acid sequence of Fc variant-SEQ ID NO. 22

| SEQ ID | |
|---|---|
| No. | Amino acid sequence |
| 22a | ASTKGPSVFPLAPSSKSTSGGTAALGCLVKDYFPEPVTVSWNSGALTSGVHTFPAVLQ |
| | SSGLYSLSSVVTVPSSSLGTQTYICNVNHKPSNTKVDKKVEPKSCDKTHTCPPCPAPE |
| | LLGGPSVFLFPPKPKDTLMISRTPEVTCVVVDVSHEDPEVKFNWYVDGVEVHNAKTKP |
| | REEQYNSTYRVVSVLNPYHQDWLNGKEYKCKVSNKALPAPIEKTISKAKGQPREPQVY |
| | TLPPSRDELTKNQVSLTCLVKGFYPSDIAVEWESNGQPENNYKTTPPVLDSDGSFFLY |
| | SKLTVDKSRWQQGNVFSCSVMHEALRWHYTQKSLSLSPGK |
| 22b | DKTHTCPPCPAPELLGGPSVFLFPPKPKDTLMISRTPEVTCVVVDVSHEDPEVKFNWY |
| | VDGVEVHNAKTKPREEQYNSTYRVVSVLNPYHQDWLNGKEYKCKVSNKALPAPIEKTI |
| | SKAKGQPREPQVYTLPPSRDELTKNQVSLTCLVKGFYPSDIAVEWESNGQPENNYKTT |
| | PPVLDSDGSFFLYSKLTVDKSRWQQGNVFSCSVMHEALRWHYTQKSLSLSPGK |
| 22c | CPPCPAPELLGGPSVFLFPPKPKDTLMISRTPEVTCVVVDVSHEDPEVKFNWYVDGVE |
| | VHNAKTKPREEQYNSTYRVVSVLNPYHQDWLNGKEYKCKVSNKALPAPIEKTISKAKG |
| | QPREPQVYTLPPSRDELTKNQVSLTCLVKGFYPSDIAVEWESNGQPENNYKTTPPVLD |
| | SDGSFFLYSKLTVDKSRWQQGNVFSCSVMHEALRWHYTQKSLSLSPGK |
| 22d | DKTHTCPPCPAPELLGGPSVFLFPPKPKDTLMISRTPEVTCVVVDVSHEDPEVKFNWY |
| | VDGVEVHNAKTKPREEQYNSTYRVVSVLNPYHQDWLNGKEYKCKVSNKALPAPIEKTI |
| | SKAKGQPREPQVYTLPPSRDELTKNQVSLTCLVKGFYPSDIAVEWESNGQPENNYKTT |
| | PPVLDSDGSFFLYSKLTVDKSRWQQGNVFSCSVMHEALRWHYTQKSLSLSPG |
| 22e | CPPCPAPELLGGPSVFLFPPKPKDTLMISRTPEVTCVVVDVSHEDPEVKFNWYVDGVE |
| | VHNAKTKPREEQYNSTYRVVSVLNPYHQDWLNGKEYKCKVSNKALPAPIEKTISKAKG |
| | QPREPQVYTLPPSRDELTKNQVSLTCLVKGFYPSDIAVEWESNGQPENNYKTTPPVLD |
| | SDGSFFLYSKLTVDKSRWQQGNVFSCSVMHEALRWHYTQKSLSLSPG |
| 22f | APELLGGPSVFLFPPKPKDTLMISRTPEVTCVVVDVSHEDPEVKFNWYVDGVEVHNAK |
| | TKPREEQYNSTYRVVSVLNPYHQDWLNGKEYKCKVSNKALPAPIEKTISKAKGQPREP |
| | QVYTLPPSRDELTKNQVSLTCLVKGFYPSDIAVEWESNGQPENNYKTTPPVLDSDGSF |
| | FLYSKLTVDKSRWQQGNVFSCSVMHEALRWHYTQKSLSLSPGK |
| 22g | APELLGGPSVFLFPPKPKDTLMISRTPEVTCVVVDVSHEDPEVKFNWYVDGVEVHNAK |
| | TKPREEQYNSTYRVVSVLNPYHQDWLNGKEYKCKVSNKALPAPIEKTISKAKGQPREP |
| | QVYTLPPSRDELTKNQVSLTCLVKGFYPSDIAVEWESNGQPENNYKTTPPVLDSDGSF |
| | FLYSKLTVDKSRWQQGNVFSCSVMHEALRWHYTQKSLSLSPG |

In one of the aspects, the Fc variant of the present invention may include non-natural amino acid(s) at one or more position. Introduction of non-natural amino acids in peptides are well known to the skilled person. Methods of making and introducing a non-naturally-occurring amino acid into a protein are known (U.S. Pat. Nos. 7,083,970; and 7,524,647). In one of the aspects, Fc variant according to the present invention has increased FcRn binding and increased half-life with modified or reduced or no ADCC and/or CDC activity. The ADCC and/or CDC activity of the Fc variant of the current invention is compared with the said activity of wild-type Fc protein or IVIg. In one of the embodiments, the Fc variant according to the present invention has amino acid substitution selected from P329G and/or M428L & N434S mutation.

Preparation of Fc Variants

Fc variant of the present invention is prepared using phage display library approach after making mutant libraries using site directed mutagenesis approach as described herein examples.

Nucleic Acid Molecules Encoding Fc Variants, Vectors and Host Cells

In one embodiment, the present invention provides nucleic acid molecules encoding the Fc variant(s) or Fc variant containing protein(s) containing as well as expression vector(s) comprising such nucleic acids and host cell(s) comprising such nucleic acid molecules encoding Fc variant from the expression vectors. Suitable vectors to produce Fc variant(s) or Fc variant containing protein(s) of the current invention by recombinant method are known in the art for the person skilled in the art. Examples of such known vectors are described in patent documents WO 2007/017903, WO 2012/046255, which are incorporated herein. The host cell according to the present invention is prokaryotic or eukaryotic cell, preferably the host cell is a mammalian cell, more preferably CHO cell.

Pharmaceutical Compositions

A pharmaceutical composition, containing one or a combination of Fc variant(s) or Fc variant containing protein(s) or Fc variant containing molecule(s) of the present invention, formulated together with a pharmaceutically acceptable carrier can be developed. Such compositions may include one or a combination of (e.g., two or more different) Fc variant(s) or Fc variant containing protein(s), or immunoconjugate(s) or bispecific molecule(s) of the invention. For example, a pharmaceutical composition of the invention can comprise a combination of Fc variant(s) or Fc variant containing protein(s) and antibody (or immunoconjugate or bispecific) that binds to different epitopes on the target antigen.

Therapeutic Uses

The Fc variant(s) or Fc variant containing protein(s) or Fc variant containing molecule(s) can be used for the treatment of diseases involving therapeutic methods that require binding of the drug to the FcRn.

In one embodiment of the present invention, the Fc variant(s) or Fc variant containing protein(s) can be used to inhibit FcRn in vivo in subjects, including humans, suffering from disease such as, but not limited to, autoimmune disease and inflammations.

In certain embodiment, the Fc variant(s) or Fc variant containing protein(s) or Fc variant containing molecule(s) used to treat disease selected from Autoimmune hemolytic anemia, Pernicious anemia, Idiopathic thrombocytopenic purpura, Goodpasture's syndrome, Bullous pemphigoid, Pemphigous vulgaris, Hashimoto's thyroiditis, Insulin-dependent diabetes mellitus (IDDM), Graves disease, Myasthenia gravis, CIDP Diabetes, Antianemics (Beta-Thalassemia), Hematopoietic Agents, Ophthalmic Drugs, Bone Diseases, Neurological Genetic Disorders, Age-related macular degeneration, Diabetic Retinopathy, Macular diseases, Non-Small Cell Lung Cancer Therapy, Ocular Genetic Disorders, Hemophilia B, Agents for (Coagulation factor IX deficiency) Cardiovascular Diseases, Type 2 Diabetes, Immunosuppressants, Rheumatoid Arthritis, Treatment of Transplant Rejection, Brain Cancer, Breast Cancer, Colorectal Cancer, Diabetic Retinopathy, Digestive/Gastrointestinal Cancer, Endocrine Cancer, Female Reproductive System Cancer, Gastric Cancer, Genitourinary Cancer, Macular diseases, Melanoma, Multiple Myeloma, Myelodysplastic Syndrome Therapy, Myeloid Leukemia Therapy, Non-Hodgkin's Lymphoma Therapy, Non-Small Cell Lung Cancer, Ovarian Cancer, Pancreatic Cancer, Prostate Cancer Therapy, Renal Cancer Therapy, Retinopathy, Aplastic anemia, Analgesic Drugs, Hematologic Genetic Disorders, Multisystem Genetic Disorders, Osteoarthritis, Scleroderma, Treatment of Autoimmune Diseases, Treatment of Gout, Urticaria, Ankylosing Spondylitis, Asthma Therapy, Dermatologic Drugs, Idiopathic Inflammatory Myopathies, Immunosuppressants, Inflammatory Bowel Disease, Interstitial Lung Diseases, Multiple Myeloma Therapy, Multiple Sclerosis, Nephritis, Psoriatic Arthritis, Systemic Lupus Erythematosus, Agents for Acute Alcoholic Hepatitis, Alzheimer's Dementia, Antiallergy/Antiasthmatic Drugs, Antiarthritic Drugs, Antipsoriatics, Breast Cancer Therapy, Cancer Associated Disorders, Dermatologic Drugs, Heart Failure Therapy, Lymphoma Therapy, Nephritis, Neurologic Drugs (Miscellaneous), Respiratory Disorders, Therapy of Inborn Errors of Metabolism. Here, the present invention is illustrated with the following non-limiting examples which should not be interpreted as limiting the scope of the invention in any way.

### EXAMPLES

The following examples are put forth so as to provide those of ordinary skills in the art with a disclosure and description of how the methods and Fc variants claimed herein are performed. They are intended to purely exemplify only and are not intended to limit the scope of the disclosure. The other Fc variants of the present invention can be developed using method as described in provided examples with some modifications. Such modifications are known to the person skilled in the art.

### Example 1: Phage Display Library Preparation

(a) Wild Fc Plasmid Generation

For library generation, the human IgG₁ Fc region containing partial sequence of hinge with CH2 and CH3 domain of heavy chain (EU221-447) along with overhangs of EcoRI and BamHI was chemically synthesized and cloned in pMA/pMK vectors.

Fc gene (SEQ ID No. 348) was isolated from these construct after digestion with EcoRI and BamHI. An amino sequence of cloned Fc gene is provided herein as SEQ ID No.: 348.

| | | | | | |
|---|---|---|---|---|---|
| DKIHTCPPCP | APELLGGPSV | ELFPPKPKDT | LMISRTPEVT | CVVVDVSHED | 270 |
| PEVKFNWYVD | GVEVHNAKTK | PREEQYNSTY | RVVSVLTVLH | QDWLNGKEYK | 320 |
| CKVSNKALPA | PIEKTISKAK | GQPREPQVYT | LPPSRDELTK | NQVSLTCLVK | 370 |
| GFYPSDIAVE | WESNGQPENN | YKTTPPVLDS | DGSFFLYSKL | TVDKSRWQQG | 420 |
| NVFSCSVMHE | ALHNHYTOKS | LSLSPGK | | | 447 |

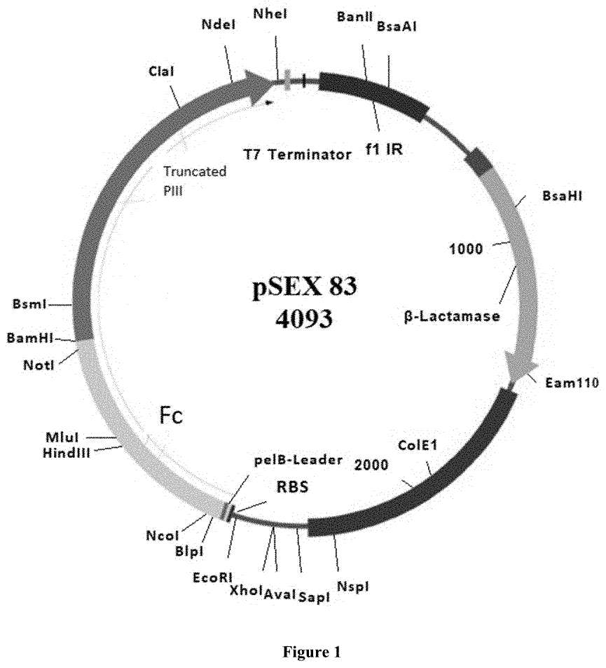

The pSEX81 (Cat No: PR3005, Progen) phagemid vector was modified in which pIII gene of phagemid vector was truncated and digested with EcoRI and BamHI and the linearized vector was ligated with the digested Fc gene. The resultant modified vector is referred herein as pSEX83. Vector map of pSEX83 is given herein as FIG. 1. The digested Fc and pSEX83 vector fragments were analyzed on agarose gel and purified from the gel using QIAquick gel extraction kit (Qiagen cat no 28706). The ligation products of both Fc (insert) and pSEX83 (vector) were transformed in electro-competent E. coli TG1 cells (Cat No: 60502-1, Lucigen). Transformed cells were plated on 2×YT agar plates containing ampicillin (100 μg/mL). The clones were analysed by EcoRI and BamHI restriction digestion and DNA sequencing using Sanger's method.

(b) Preparation of Secondary Libraries for Affinity Maturation

Two different strategies were followed to prepare Fc variant phage display library.

Strategy 1: PCR Primer Based Site Directed Mutagenesis

Four different regions in the CH2-CH3 (EU250-259, EU307-311, EU375-381 and EU432-437) domains of the Fc gene were targeted for introducing random mutations and preparing phage display library.

The mutations in the selected regions were introduced by using PCR with respective primer pairs (RK173/RK174, RK175/RK176, RK177/RK178, RK179/RK180, RK181/RK182 and RK183/RK184) having SapI restriction site (Table 4). Each primer pair was designed in such a way that they amplified a complete plasmid in a linear form which after restriction digestion by SapI followed by ligation with T4 DNA ligase resulted in a circular plasmid with mutations introduced by the primer pair. Each pair was used to make a separate library. Also DNA from one library was used to make libraries for adding mutations to other regions.

In brief, 100 ng of the template DNA of Fc gene in pSEX83 vector was used for amplification using the primer pairs mentioned in table 5. Once the template was amplified using the randomized primers the product was digested with SapI to obtain sticky ends. After PCR purification, the digested product was ligated overnight at 16° C. Ligated DNA was then transformed to freshly prepared electrocompetent cells (TG1). Transformed cells were cultured for the production of phages. The phage production was done separately for each library as explained earlier (Brockmann et al., 2011).

TABLE 5
list of primers used in PCR Primer based site
directed mutagenesis

| | |
|---|---|
| RK173 | ACTATCGTTAGCTCTTCTGATNNSNNSNNSNNSTCACGCACCC |
| | CTGAGGTTAC |
| RK174 | ACTATCGTTAGCTCTTCTATCTTTCGGCTTTGGCGGAAAAAGG |
| | AACACTGAAGG |
| RK175 | ACTATCGTTAGCTCTTCTATGNNSNNSNNSNNSCCTGAGGTTA |
| | CCTGCGTTGTTGTT |
| RK176 | ACTATCGTTAGCTCTTCTCATCAGTGTATCTTTCGGCTTTGGC |
| | GGAAAAAGGAA |
| RK177 | ACTATCGTTAGCTCTTCTTTANNSNNSNNSCATCAGGACTGGC |
| | TGAATGGAAAA |
| RK178 | ACTATCGTTAGCTCTTCTTAAGACTGATACCACGCGATAGGTG |
| | GAATTATATTG |
| RK179 | ACTATCGTTAGCTCTTCTCCGNNSNNSNNSNNSGTTGAATGGG |
| | AGTCCAATGGC |
| RK180 | ACTATCGTTAGCTCTTCTCGGGTAGAATCCCTTAACAAGGCAA |
| | GTCAATGA |
| RK181 | ACTATCGTTAGCTCTTCTATTNNSNNSNNSNNSGAGTCCAATG |
| | GCCAACCAGAGAAC |
| RK182 | ACTATCGTTAGCTCTTCTAATGTCTGACGGGTAGAATCCCTTA |
| | ACAAGGCAAGT |
| RK183 | ACTATCGTTAGCTCTTCTGCTNNSNNSNNSNNSTATACGCAGA |
| | AGAGTCTTAGTTTG |
| RK184 | ACTATCGTTAGCTCTTCTAGCCTCATGCATTACGGAGCATGAG |
| | AACACGTTCCC |

Strategy 2: Kunkel Mutagenesis and sRCA

Kunkel mutagenesis was performed with sRCA following reference 9. Small culture of CJ236 cells was infected with phages carrying plasmid of the Fc gene. The infected cells were grown in 2×YT containing uridine (6 μg/mL) and carbenicillin antibiotic (100 μg/mL). Phages were produced after superinfection with VCS M13 helper phage (Agilent technologies, USA) and purified by precipitating with PEG6000 (4%) and NaCl (500 mM). Single-stranded uridylated DNA (ss (U) DNA), was extracted from the purified phages by using M13 purification kit from E.Z.N.A.® M13 DNA mini kit (OMEGA bio-tek, USA) following the manufacturer's instructions.

This ss (U) DNA was used as a template in Kunkel mutagenesis and different primers with random mutations were used for making the library. Primers targeting different regions of Fc (Table 6) were hybridized separately to the ss (U) DNA template and extended by Kunkel mutagenesis method. The product was UDG-treated and selectively amplified by RCA (9). The RCA product was digested with Hind III, circularized using T4 DNA ligase and transformed in SS320 cells. Phages (the secondary library) were produced separately for each mutagenesis primer by superinfection with helper phage as described below.

TABLE 6
list of primers used in Kunkel mutagenesis

| | |
|---|---|
| RK114 | GGTAACCTCAGGGGTGCGTGASNNSNNSNNSNNATCTTTCGGC |
| | TTTGGCGGAAA |
| RK115 | AACAACGCAGGTAACCTCAGGSNNSNNSNNSNNCATCAGTGTA |
| | TCTTTCGGCTT |
| RK116 | GACATCAACAACAACGCAGGTSNNSNNSNNSNNGCGTGAAATC |
| | ATCAGTGTATC |
| RK117 | TCCATTCAGCCAGTCCTGATGSNNSNNSNNTAAGACTGATACC |
| | ACGCGATA |
| RK118 | CTCTTTTCCATTCAGCCAGTCSNNSNNSNNGACAGTTAAGACT |
| | GATACCAC |
| RK119 | CTCTTTTCCATTCAGCCAGTCSNNSNNSNNSNNSNNTAAGACT |
| | GATACCACGCGATA |
| RK120 | ATTGGACTCCCATTCAACCGCSNNSNNSNNCGGGTAGAATCCC |
| | TTAACAAG |
| RK121 | CTCTGGTTGGCCATTGGACTCSNNSNNSNNCGCAATGTCTGAC |
| | GGGTAGAA |
| RK122 | ATAGTGATTATGCAAAGCCTCSNNSNNSNNGGAGCATGAGAAC |
| | ACGTTCCC |
| RK123 | ACTAAGACTCTTCTGCGTATASNNSNNSNNSNNAGCCTCATGC |
| | ATTACGGAGCA |
| RK124 | CGACAAACTAAGACTCTTCTGSNNSNNSNNSNNATGCAAAGCC |
| | TCATGCATTAC |

### Example 2: Mutant Fc Phage Production from PCR and Kunkel Library and Purification Thereof

Flasks containing 2×YT media with carbenicillin or ampicillin (100 μL/mL) were inoculated with the cells from the glycerol stock of the above described library at the initial concentration of 0.06 OD₆₀₀. Cultures were grown at 37° C. with shaking at 250 rpm till they reach an OD₆₀₀ of up to ˜0.4-0.6. Helper phages either, VCSM13 (Agilent, Cat no. 200251) or M13KO7 (GE healthcare, Cat no. 27152401), were then added to the culture at a multiplicity of infection (MOI) of 20, and incubated first without shaking at 37° C. for 40 minutes, followed by another 40 minutes of incubation at 37° C. with shaking. Kanamycin was then added to the media and culture was grown overnight at 26° C. at 150 rpm. Overnight phage culture was centrifuged at 4000 g and cell pellet was discarded. PEG (20%)/NaCl (2.5 M) solution at a ratio of 1:5 was added to the supernatant in order to precipitate the phages. Resuspended solution was incubated on ice for 20 minutes followed by centrifugation at 14,000 g for 15 minutes at 4° C. Supernatant was discarded and pellet was resuspended in 1 mL of sterile PBS with 0.01% sodium azide. Phages were stored at 4° C. until further use.

### Example 3: Biopanning of High Affinity Fc Variants Expressing Phages

Fc mutant library (1×10¹² PFU) prepared in example 2 was screened for high affinity Fc binders to FcRn receptor. First round of panning against FcRn/FcGRT antigen (Sino biological, Cat No, CT071-H27H) at pH 6.0, was performed using antigen-immobilized, Immunotubes (Quidel, USA) that were prepared by incubating them with a 5 μg/mL FcRn protein solution in carbonate buffer (0.1 M, pH 9.6) overnight at 4° C. Immunotubes were washed 3 times with PBS and then incubated with phages in PBS for 2 h at 25° C. with constant rotation. The tubes were washed ten times with 4 mL PBS with tween 20 (0.1%) and subsequently 10 times with PBS. The bound phages were eluted with glycine-HCL pH 2.1 (0.1 M). The eluted phages were rescued by infection of TG1 E. coli cells, plated, and phages produced as described in example 3.

Second and third round of panning were performed on the FcRn antigen coated immunotubes in such a manner that output phages from first round of panning (1×10¹¹ CFU) and second (1×10¹⁰ CFU) round of panning were used, as input phages for second and third round of panning, respectively. Phages after third round of panning were infected in TG1 cells as mentioned before and phagemid DNA was isolated for Fc variant cloning in suitable expression vector.

### Example 4: Screening of Individual Fc Mutant Clones as Soluble Fc Proteins

Cloning to Expression Vector and Protein Production

The Fc variant genes from the enriched library produced after 3 rounds of panning were cloned into the expression vector pOPE101 (carbenicillin resistant) (Progen, Germany) or its modified version pOPE102 (kanamycin resistant) such that the individual Fc mutant clones could be produced as HIS-tagged fusion products. Both the vector DNA (pOPE101 or pOPE102) and phagemid DNA were digested with restriction enzymes (EcoRI and BamHI) to isolate vector and Fc variant genes, respectively. Both restriction digested vector and Fc gene were ligated and transformed to TG1 electrocompetent cells. Transformed cells were plated on to the 2×YT agar plates containing carbenicillin antibiotic. Individual clones were picked from the 2×YT agar plates and cultured in 15 mL tube with 5 mL of 2×YT media containing carbenicillin or ampicillin (100 μL/mL) overnight at 32° C. at 200 rpm. Next day, cultures were reinoculated to fresh 2×YT medium containing carbenicillin (100 μg/mL) and glucose (0.1%) at a volumetric ratio of 1:200. Cultures were grown until the OD₆₀₀ reached ˜0.6-0.8. After that 1 mM Isopropyl β-D-1-thiogalactopyranoside (IPTG) was added to the culture and grown overnight at 30° C. at 200 rpm. Overnight culture was spun at 10,800 g for 15 minutes and supernatant was removed. Cell pellet was resuspended in 1/20^{th} volume of the original culture in 1×PBS buffer pH-7.4 containing 2 mg/mL lysozyme, 0.1% triton X and 1 U/100 mL benzonase and incubated at 37° C. for 1 h. Resuspended pellet was centrifuged at 10,800 g for 15 minutes and supernatant was collected as the cell lysate containing the soluble His-tagged Fc variants. This cell lysate fraction was used in immunoassays to study FcRn-binding after quantifying the soluble Fc in the periplasmic fraction.

Quantification of Soluble Fc in Periplasmic Fraction

Clones were cultured individually for the production of soluble Fc variants as described above. Total Fc in cell lysate or periplasmic fraction was quantified by immobilizing the soluble Fc on maxisorp plates pre-coated with mouse anti-Human IgG antibody (Sigma, Cat no. 16760). Coating was done overnight with the mouse anti-Human IgG antibody at concentrations of 2.5 μg/mL (100 μL/well) in coating buffer (0.1 M NaHCO₃, pH 9.6). After washing the plate twice, cell lysate 100 μL ( 1/20 v/v in 1×PBS) was added and incubated for 1 h at 25° C. Known concentrations of serially diluted wild Fc IgG1 were used as standards for the calculation of amount of Fc protein in cell lysate. The plate was washed 3 time with PBST (0.1% Tween 20 in 1×PBS). 100 μL (1:10000) of HRP conjugated goat anti-human IgG Fc, F(ab)′2 Fragment (Invitrogen, cat no. A24476) was added to each well of the plate and incubated for 1 h. Plate was then washed 5 times with PBST followed by addition of substrate o-phenylenediamine dihydrochloride (OPD) (100 μL/well) for 15 minutes at 37° C. The reaction was stopped using 1N H₂SO₄ (100 ul/well) and optical density (OD) was measured at 450 nm using TECAN INFINITE® M1000pro. Concentration of Fc in cell lysate was then calculated using reference standards.

Qualitative Analysis of Fc Variants

FcRn antigen was coated on polystyrene plate (100 ng/100 μL/well), overnight at 4° C. in coating buffer (0.1 M NaHCO₃, pH 9.6). After washing the plate twice, 100 ng of Fc variant diluted in 100 μL PBS pH 6.0 was added to each well and incubated for 1 h at 25° C. Plates were washed 4 times with PBST (0.1% Tween 20 in 1×PBS pH 6.0) followed by addition of 100 μL (1:10000) of HRP conjugated goat anti-human IgG Fc, F(ab)′2 Fragment (Invitrogen, cat no. A24476) and incubated for 1 h at 25° C. Plate was then washed 5 times with PBST followed by addition of substrate o-phenylenediamine dihydrochloride (OPD) (100 μL/well) for 15 minutes at 37° C. The reaction was stopped using 1N H₂SO₄ (100 μL/well) and optical density was measured at 450 nm using TECAN INFINITE® M1000pro. Individual clones with relatively high OD signal compared to the wild Fc was shortlisted for further characterization.

### Example 5: Determination of Kinetic Rate Constants of Fc Variants Binding to Recombinant Human Neonatal Fc Receptor (rhFcRn)

The kinetic constants for the binding of Fc variants to recombinant human neonatal Fc receptor (rhFcRn) were determined by Surface Plasmon Resonance-based measurement using the ProteOn™ XPR36 (Bio-Rad). The rhFcRn receptor (Sino Biologics) was immobilized on a GLC chip following manufacturer's instruction. 10 mM phosphate buffer saline (PBS) (10 mM phosphate buffer, pH 6.0, 150 mM NaCl, with 0.005% Tweet 20) was used as running buffer to carry out the kinetic measurements. To measure the association rate constant (Kₐ) and dissociation rate constant (K_{d}), five dilutions of Fc variants were prepared in above mentioned running buffer and injected at a flow rate of 100 μL/min with an association time of 180 s and dissociation time of 600 s. Reactions were carried in PBS (pH 6.0, 0.005% surfactant P20) at 25° C. After each sample run, the chip surface was regenerated using two pulses of PBS (0.005% surfactant P20), pH 7.4, followed by one pulse of glycine buffer pH 1.5. The data, in the form of sensograms, was analysed using the data-fitting programs in the ProteOn™ system. The kinetic constants of rhFcRn binding to different Fc variants are shown in table 7.

TABLE 7
Kinetic constants for selected FcRn binders

| SEQ ID No. | Sample code | Kₐ | K_{d} | K_{D} |
|---|---|---|---|---|
| 3 | A4 | 1.12E+06 | 1.98E−02 | 1.08E−08 |
| 5 | D9-6 | 3.42E+05 | 2.93E−03 | 8.60E−09 |
| 6 | H11_4-2 | 1.06E+06 | 1.07E−02 | 1.02E−08 |
| 10 | B9-10 | 5.26E+06 | 1.56E−02 | 2.98E−09 |
| 19 | A9-10 | 4.21E+06 | 1.17E−02 | 2.79E−09 |
| 20 | F3-9 | 7.27E+06 | 2.64E−02 | 3.63E−09 |
| 22 | H11 + A4 | 7.51E+06 | 2.16E−03 | 2.88E−10 |

### Example 6: Determination of Kinetic Rate Constants of Fc Variants Binding to Recombinant Human Neonatal Fc Receptor (rhFcRn) at Different pH Condition

In this experiment, affinity constants for the binding of Fc variants to recombinant human neonatal Fc receptor (rhFcRn) were determined by Surface Plasmon Resonance-based measurement using the ProteOn™ XPR36 (Bio-Rad) at two different conditions. 10 mM phosphate buffered saline (PBS) (10 mM phosphate buffer, 150 mM NaCl, with 0.005% Tween 20) was used as the running buffer to carry out the kinetic measurements. To measure the affinity constant, five dilutions of Fc variants were prepared and injected at a flow rate of 100 μL/min with an association time of 180 s and dissociation time of 600 s. All the reactions were carried at 25° C. Samples were analyzed for FcRn binding in different pH conditions to check the effect of pH on FcRn binding and dissociation of molecule from FcRn. Affinity constants for Fc variant having sequence as set forth in SEQ ID No. 22 (H11+A4) to rhFcRn was measured. The buffer for association phase of interaction is the sample dilution buffer and buffer for dissociation phase of interaction is the running buffer. In condition 1, buffer for association phase was PBST pH 6.0, while buffer for dissociation phase was PBST pH 7.4. In condition 2, buffer for association phase was PBST pH 7.4, while buffer for dissociation phase was PBST pH 6.0 Fc variants used to conduct this experiment was produced in CHO cell line. The affinity constants of rhFcRn binding to Fc variant at two different pH conditions are shown in table 8.

TABLE 8
Affinity constant of FcRn binder at two different pH conditions

| SEQ ID | | | |
|---|---|---|---|
| No. | Sample code | Condition | K_{D} |
| 22 | H11 + A4 | Condition 1 | 4.98E−09 |
| | | (Association in PBST pH 6.0, | |
| | | Dissociation in PBST pH 7.4) | |
| 22 | H11 + A4 | Condition 2 | 8.44E−08 |
| | | (Association in PBST pH 7.4, | |
| | | Dissociation in PBST pH 6.0) | |

As it can be seen from table 8, Fc variant of the current invention can block FcRn with higher affinity at pH 6.0 as compared to at pH 7.4. Thus, the Fc variant of the current invention has retention of pH dependence (higher affinity at pH 6.0 than at near-neutral pH) characteristic of FcRn interactions. Therefore, Fc variant prepared according to the current invention can reduce total serum IgG levels in the circulation.

### Example 7: Determination of Kinetic Rate Constants of Fc Variants Binding to Recombinant Neonatal Fc Receptor (rFcRn) of Different Species

In this experiment, binding of the Fc variant of the current invention to FcRn of different species was determined by surface plasmon resonance-based measurement using the ProteOn™ XPR36 (Bio-Rad). To check kinetic rate constants, the experiment was conducted using recombinant FcRn of mouse, monkey and human with Fc variants having sequence as set forth in SEQ ID No. 22 (H11+A4). Fc variant used to conduct this experiment was produced using CHO cell as an expression system. FcRn (of different species) was immobilized on a GLC sensor chip surface using a standard amine coupling chemistry, and essentially, following the manufacturers' instruction. 10 mM phosphate buffered saline (PBS) (10 mM phosphate buffer, pH 6.0, 150 mM NaCl, 0.005% Tween 20) was used as the running buffer to carry out the kinetic measurements. To measure the association rate constant (k_{assoc}) and dissociation rate constant (k_{dissoc}), five dilutions of Fc muteins were prepared in above mentioned running buffer and injected at a flow rate of 100 μL/min with an association time of 180 s and dissociation time of 600 s. All the reactions were carried at 25° C. After each sample run, the chip surface was regenerated using PBST, pH 7.4. The data, in the form of sensorgrams, was analyzed using the data-fitting programs in the ProteOn™ system.

The kinetic constants of FcRn (of different species) binding to Fc variant are shown in table 9.

TABLE 9
Kinetic constant of FcRn binder for FcRn of different species

| Type of FcRn | Kₐ | K_{d} | K_{D} |
|---|---|---|---|
| Human FcRn | 4.10E+06 | 3.24E−03 | 7.91E−10 |
| Mouse FcRn | 1.18E+06 | 7.24E−04 | 6.15E−10 |
| Monkey FcRn | 4.42E+06 | 3.54E−03 | 8.01E−10 |

As it can be seen from table 9, Fc variant of the current invention can block not only human FcRn, but also mouse FcRn as well as monkey FcRn in-vitro. The advantageous characteristics of cross-reactivity with monkey FcRn means that the Fc variant of the current invention can be tested in non-human primates and the resulting data can be very useful in predicting their pharmacokinetics, pharmacodynamics and toxicity of the Fc variant of the current invention in humans.

### Example 8: Determination of Kinetic Rate Constants is of Fc Variants Binding to Fc Gamma Receptors

In this experiment, the kinetic constants for the binding of Fc variant to recombinant Fc gamma receptors were determined by Surface Plasmon Resonance-based measurement using the ProteOn™ XTR36 (Bio-Rad). For this experiment, FcγRIIIa (Phe) and FcγRIIIa (Val) Fcγ receptors were analysed for binding to Fc variant of the current invention having sequence as set forth in SEQ ID No. 22(H11+A4). All the recombinant human Fc receptors were directly immobilized on a GLC sensor chip surface using a standard amine coupling chemistry, and essentially, following the manufacturers' instruction. 10 mM phosphate buffered saline (PBS) (10 mM phosphate buffer, pH 7.4, 150 mM NaCl, with 0.005% Tween 20) was used as the running buffer to carry out the kinetic measurements for all the Fc receptor. To measure the association rate constant (k_{assoc}) and dissociation rate constant (k_{dissoc}), five dilutions of Fc variants were prepared in above mentioned running buffer and injected at a flow rate of 50 μL/min. Association time and dissociation time at each interaction is mentioned in table below. All the reactions were performed at 25° C. The data, in the form of sensorgrams, were analyzed using the data-fitting programs available with the ProteOn™ system.

TABLE 10
Experiment details for Fc receptors binding Fc variants

| Receptor | Association time (s) | Dissociation time (s) |
|---|---|---|
| FcγRIIIa (Phe) | 240 | 600 |
| FcγRIIIa (Val) | 240 | 600 |

The Fc variant used to conduct this experiment was produced in CHO cell line. Control molecule representing human IgG₁ control (lacking substitutions of the present invention) was kept to determine level of affinity of Fc variant of the current invention towards Fc gamma receptors.

TABLE 11
Kinetic constant of Fc variant to Fc gamma receptors

| Type of Fc | | | | |
|---|---|---|---|---|
| gamma receptor | Sample code | Kₐ | K_{d} | K_{D} |
| FcγRIIIa (Phe) | H11 + A4 | 3.15E+05 | 7.62E−02 | 2.41E−07 |
| | IgG₁ control | 1.27E+05 | 2.80E−01 | 2.20E−06 |
| FcγRIIIa (Val) | H11 + A4 | 1.62E+05 | 3.33E−02 | 2.05E−07 |
| | IgG₁ control | 4.79E+04 | 6.13E−02 | 1.28E−06 |

As it can be seen from table 11, Fc variant of the current invention can block FcγRIIIa (including allotypes V158 and F158) with high affinity as compared to a molecule representing IgG₁ control. This illustrated activity of Fc variant of the current invention of blocking Fc gamma receptors shows that Fc variant of the current invention can inhibit immune-complex mediated FcγR activation.

### Example 9: Construction of Monomer and Multimer (Dimer) DGV-H11A4 of Fc Variant Having SEQ ID No. 22

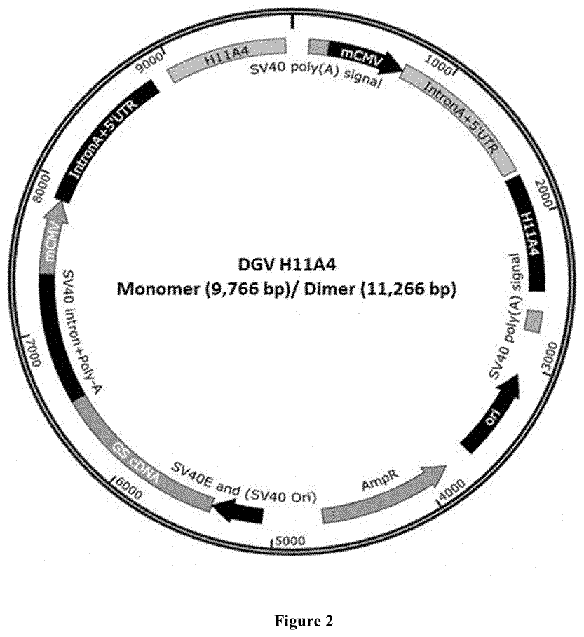

Chemically synthesized gene of the Fc variant monomer having amino acid sequence of SEQ ID NO. 22 (H11+A4; It can also be referred as H11A4.), and Fc dimer, having amino acid sequence of SEQ ID NO. 22 (H11+A4; It can be referred as H11A4.), in which two chains of Fc variant monomer were linked with Glycine-serine linker (SEQ ID NO. A-GGGSGGGSGGGSGGGSSGGGSS) and cysteine residue at EU position 226 and 229 of second chain, were changed to serine to prevent self-dimerization were, obtained in pMK vector were obtained from Geneart, Germany. Fc variant monomer and dimer genes were isolated from pMK Geneart constructs by restriction digestion with HindIII and EcoRI. The HindIII and EcoRI digested Fc variant monomer H11A4 gene of ˜0.7 kb and Fc variant dimer of ˜1.5 kb were individually ligated to HindIII and EcoRI digested pXC-17.4 vector (Lonza) and pXC-18.4 vector (Lonza). The ligation products were transformed in E. coli Top10F′ and transformants were scored on the basis of ampicillin resistance. The clones were analysed by restriction digestion with HindIII and EcoRI. A positive clone from each pXC-17.4 H11A4 monomer and dimer and pXC-18.4 H11A4 monomer and dimer were digested with SalI and NotI. The digested products were resolved on 1% agarose gel. The final DGV H11A4 monomer, carrying two expression assemblies were prepared by ligating ˜6.9 kb fragment from pXCH11A4 monomer and ˜2.7 kb fragment from pXC-18.4 H11A4 monomer. Similarly, the final DGV H11A4 dimer carrying two expression assemblies, was prepared by ligating ˜7.7 kb fragment from pXCH11A4 dimer and ˜3.5 kb fragment from pXC-18.4 H11A4 dimer. The ligation products were transformed in E. coli Top 10F′ and transformants were scored based on the ampicillin resistance. Final DGV-H11A4 monomer and dimer vectors were confirmed by restriction digestion characterization and Sanger sequencing. The vector was linearized with PvuI for transfection in CHO-GS cells (Lonza). A map of the vector used for the preparation of Fc monomer and Fc dimer is given here as FIG. 2.

### Example 10: Construction of Full-Length Antibody DGV-H11A4 with Fc Variant Having SEQ ID No. 22

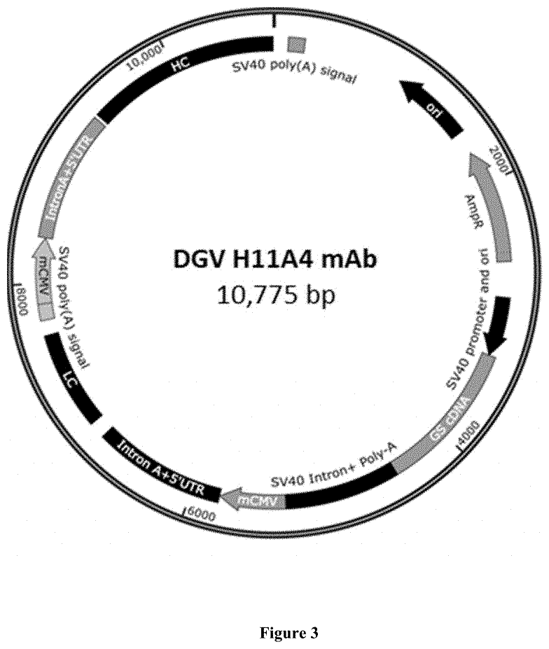

Fc variant H11A4 was prepared as full-length monoclonal antibody molecule by joining the Fc region with unrelated (not cross reactive to human/mice/NHP) heavy chain variable domain in combination with unrelated (not cross reactive to human/mice/NHP) light chain. Fc variant H11A4 region was amplified to insert CH1 domain and ApaI restriction site at 5′ end and EcoRI restriction site at 3′ end. This ˜1.0 kb insert was digested with ApaI and EcoRI to facilitate cloning in frame with heavy chain variable region. The MluI and ApaI digested variable region of heavy chain, gene of ˜0.5 kb and ApaI-EcoRI digested H11A4 PCR fragment was ligated to MluI and EcoRI digested DGV vector harbouring light chain gene. Ligation product was transformed into E. coli Top10F′ cells and transformants were scored based on the ampicillin resistance. Final DGV-H11A4 mAb vector was confirmed by restriction digestion characterization and Sanger sequencing. The vector was linearized with PvuI for transfection in CHO-GS cells (Lonza). A map of the vector used for the generation of full-length antibody comprising Fc monomer is given here as FIG. 3.

### Example 11: Generation of Cell Lines Expression Fc Constructs

This example describes the generation of stable transfected cell lines expressing Fc variants. All vector constructs as described in examples 9 and 10 were used for transfections. Plasmids were linearized with PvuI restriction enzyme prior to transfection. Chinese Hamster ovary (CHO), which is one of the suitable hosts for expression of recombinant proteins, was used for cell line generation. CHO cells were seeded ˜24 hours prior to transfection at a density of 0.5 million/mL to have cells in the exponential phase. Transfections were performed using Neon Transfection system (Invitrogen) by electroporation technique following manufacturer's instructions. Post-transfection, cells were plated in 24 well cell culture plates containing 1 mL of pre-warmed ProCHO5 Serum Free media (Lonza, Switzerland) containing selection pressure and incubated in a humidified incubator at 37° C. in presence of 5% CO₂. The cell numbers of all the transfected pools were regularly monitored and regular media exchanges were given. Once the cells recovered from transfection, cells were further expanded to 6 well culture plates, T-flasks and culti-tubes (TPP).

Fed-batch cultures were performed for transfected pools of all Fc variant candidates in culti-tube (TPP) for recombinant protein production. Cells were seeded at a density of 0.3×10⁶ cells/mL in ActiPro production medium from Hyclone, GE. Culti tubes were incubated in a humidifed Kuhner shaker at 37° C. temperature, 5% CO₂ level with shaking speed of 230 RPM. A fixed daily feeding regimen was followed during the culture for all the pools using chemically defined feeds from Hyclone, GE. After 72 hours of culture, feeding was initiated and continued till the batch was harvested.

Upon harvest, the culture supernatants were collected for protein purification by Protein A affinity chromatography. These purified protein candidates were further tested for various in-vitro assays as described in above examples. It has been observed that Fc variant (SEQ ID No. 22) in multimeric form and Fc variant in full-length antibody form has technical effect similar to Fc monomer. Thus, the Fc variant of the current invention can achieve the same technical effect in all the constructed form (monomer, multimer and full-length antibody).

### Example 12: Effect of Fc Variant on Total Serum IgG Levels in Wild-Type C57BL/6 Mice

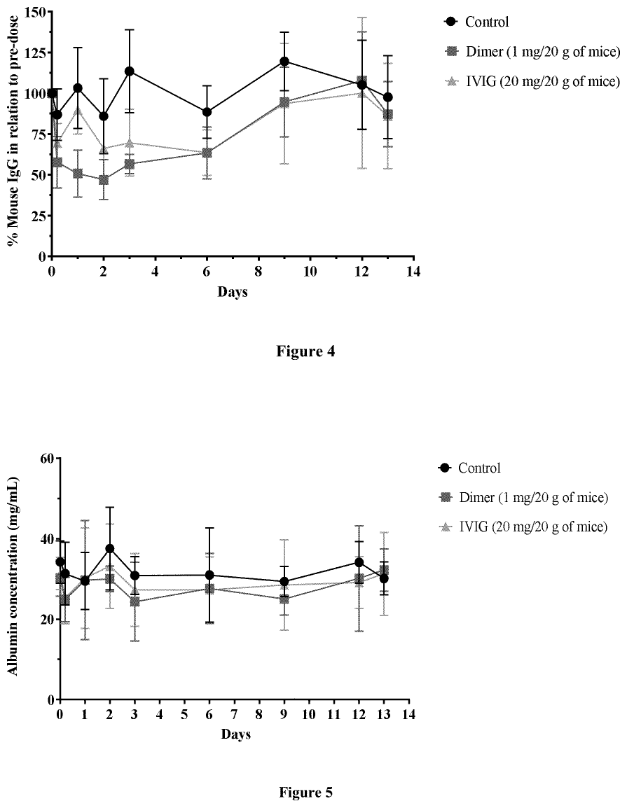

In the present study, Fc dimer prepared according to example 9 was compared to commercially available IVIg to determine the effect of Fc variant of the present invention on total serum IgG level in circulation. Wild-type C57BL/6 mice were randomized based on their body weight. At zero time point (Pre-dose), blood collection was performed. Within 1 hour, 50 mg/kg of Fc dimer and 1 g/kg of IVIg were administered via intra-venous route to respective group of study animals (6 mice per group). Blood samples were collected at time points—5 h, 24 h (Day 1), 48 h (Day 2), 72 h (Day 3), 144 h (Day 6), 216 h (Day 9), 288 h (Day 12) and 312 h (Day 13). Endogenous IgG levels were determined using ELISA from the collected samples at mentioned time points. The results in graphical presentation are shown in FIG. 4. Both IVIg and Fc dimer showed a reduction in endogenous mouse IgG levels as compared to control, with Fc dimer showing greater IgG clearance as compared to IVIg till day 6. Lowest % IgG reduction of ˜47% was observed with Fc dimer as compared to ˜66% with IVIg in relation to pre-dose IgG. Albumin levels were also determined at the mentioned time points. Overall, no differences were observed across all time points and groups (FIG. 5). Fc dimer prepared according to the current invention did not impact the albumin levels in serum. It evident that the Fc variant prepared according to the current invention does not interfere with the binding site of albumin on FcRn and allow FcRn to maintain albumin level in serum. Similar experiment was conducted following the same process as described herein example 12 for full-length antibody containing Fc variant of the current invention as well as monomeric form of Fc variant of the current invention having SEQ ID No. 22.

### Example 13: Effect of Fc Variant on Fc Gamma Receptors Dependent ADCC Activity

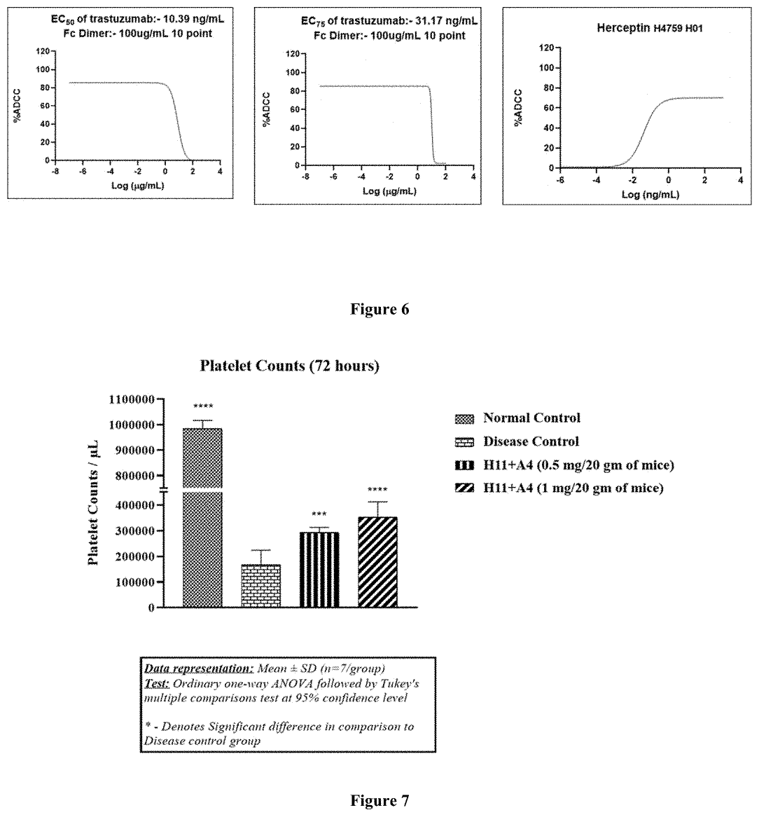

SKBR3 cells were stained with Calcein-AM dye, seeded with trastuzumab at EC₅₀ and EC₇₅ concentration of 10.39 ng/mL and 31.17 ng/mL respectively, and incubated for 30 min at 37° C. and 5% CO₂. To determine whether Fc dimer prepared according to example 9 could block ADCC, serially diluted Fc dimer starting from 100 μg/mL and human PBMC at 1:30 ratio of target to effector cells were added to the wells containing SKBR3 cells and incubated for 4 hrs at 37° C. and 5% CO₂. Following incubation, the plates at 2000 rpm was centrifuged for 5 minutes at 25° C. and 100 μL supernatant was collected and transferred it to 96 well black clear bottom plate. Reading was taken in plate reader at excitation 494 nm and emission at 515 nm, with cut off set at 515 nm. Results are shown in FIG. 6. As it can be seen in FIG. 6, Fc dimer of the current invention can reduce ADCC activity mediated by trastuzumab confirming that by its stronger binding to Fc gamma receptors, it can prevent the binding of trastuzumab to NK cells present in PBMCs.

### Example 14: Effect of Fc Variant in Chronic Idiopathic Thrombocytopenia Purpura (ITP) Animal Model

In the present study, the therapeutic potency of H11+A4 (SEQ ID NO. 22) was tested in a mouse model of acute immune thrombocytopenia. Specifically, C57BL/6 mice were randomized based on their body weight and subsequently treated with two different doses of H11+A4 (i.e., 0.5 mg/20 gm of mice & 1 mg/20 gm of mice) or with phosphate buffered saline via intravenous injection (7 animals/group). One hour later, mice were treated with anti-platelet antibody MWReg30 (5 μg/20 gm of mice) or with phosphate buffered saline via intravenous route. After 24 hours, once again mice were treated with MWReg30 (5 μg/20 gm of mice) or with phosphate buffered saline via intraperitoneal route. Blood samples were collected for determination of platelet counts at 72 hours after 1^{st} injection of MWReg30. Platelet counts were determined via haematology analyser from collected samples & plotted. Result is shown in FIG. 7. As it can be seen from FIG. 7, pre-treatment with H11+A4 reduced MWReg30 (anti-platelet antibody) induced thrombocytopenia at both the dose levels in dose dependant manner & molecule has significant potential to ameliorate thrombocytopenic condition.

### REFERENCES INCORPORATED IN CURRENT PATENT APPLICATION

1. Leona E. Ling 1, Jan L. Hillson, M281, an Anti-FcRn Antibody: Pharmacodynamics, Pharmacokinetics, and Safety Across the Full Range of IgG Reduction in a First-in-Human Study, Clinical Pharmacology & Therapeutics, Volume 105 Number 4, April 2019
2. Peter Kiessling et al., The FcRn inhibitor rozanolixizumab reduces human serum IgG concentration: A randomized phase 1 study, Sci. Transl. Med. 9, eaan1208, November 2017
3. T. Sockolosky, F. C. Szoka, The neonatal Fc receptor, FcRn, as a target for drug delivery and therapy, Adv. Drug Deliv. Rev., 91, 109-124, 2015
4. Deisenhofer, Crystallographic refinement and atomic models of a human Fc fragment and its complex with fragment B of protein A from Staphylococcus aureus at 2.9- and 2.8-.ANG. resolution, Biochemistry 20:2361-2370, 1981
5. Edelman, G. M. et al., The covalent structure of an entire gamma G immunoglobulin molecule. Proc. Natl. Acad. USA, 63, 78-85, 1969
6. Roux et al., Comparisons of the Ability of Human IgG3 Hinge Mutants, IgM, IgE, and IgA2, to Form Small Immune Complexes: A Role for Flexibility and Geometry, J. Immunol. 161: 4083, 1998
7. Jefferis et al., Interaction sites on human IgG-Fc for FcgR: current models, Immunol Lett., 82:57-65, 2002
8. Angal S, King D J, Bodmer M W, Turner A, Lawson A D, Roberts G, Pedley B, Adair J R. A single amino acid substitution abolishes the heterogeneity of chimeric mouse/human (IgG4) antibody. Mol Immunol. 30(1): 105-8, 1993.
9. Huovinen T, Brockmann E-C, Akter S, Perez-Gamarra S, Yla″-Pelto J, et al., Primer Extension Mutagenesis Powered by Selective Rolling Circle Amplification., Volume 7, Issue 2, e31817, 2012

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. An Fc variant comprising combination of substituting amino acids at specific EU position in wild-type Fc protein wherein said Fc variant binds with high affinity to human FcRn relative to the wild-type Fc protein and comprising amino acid sequence selected from SEQ ID NO. 1, SEQ ID NO. 2, SEQ ID NO. 3, SEQ ID NO. 4, SEQ ID NO. 5, SEQ ID NO. 7, SEQ ID NO. 9, SEQ ID NO. 11, SEQ ID NO. 14, SEQ ID NO. 15, SEQ ID NO. 18, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 29, SEQ ID NO. 30, SEQ ID NO. 31, SEQ ID NO. 32, SEQ ID NO. 34, SEQ ID NO. 35, SEQ ID NO. 36, SEQ ID NO. 38, SEQ ID NO. 39, SEQ ID NO. 40, SEQ ID NO. 41, SEQ ID NO. 42, SEQ ID NO. 43, SEQ ID NO. 44, SEQ ID NO. 45, SEQ ID NO. 46, SEQ ID NO. 47, SEQ ID NO. 48, SEQ ID NO. 49, SEQ ID NO. 50, SEQ ID NO. 51, SEQ ID NO. 52, SEQ ID NO. 53, SEQ ID NO. 54, SEQ ID NO. 55, SEQ ID NO. 56, SEQ ID NO. 57, SEQ ID NO. 58, SEQ ID NO. 59, SEQ ID NO. 60, SEQ ID NO. 61, SEQ ID NO. 62, SEQ ID NO. 63, SEQ ID NO. 64, SEQ ID NO. 65, SEQ ID NO. 66, SEQ ID NO. 67, SEQ ID NO. 68, SEQ ID NO. 69, SEQ ID NO. 70, SEQ ID NO. 71, SEQ ID NO. 72, SEQ ID NO. 73, SEQ ID NO. 74, SEQ ID NO. 75, SEQ ID NO. 76, SEQ ID NO. 77, SEQ ID NO. 78, SEQ ID NO. 79, SEQ ID NO. 80, SEQ ID NO. 81, SEQ ID NO. 82, SEQ ID NO. 83, SEQ ID NO. 84, SEQ ID NO. 85, SEQ ID NO. 86, SEQ ID NO. 87, SEQ ID NO. 88, SEQ ID NO. 89, SEQ ID NO. 90, SEQ ID NO. 91, SEQ ID NO. 92, SEQ ID NO. 93, SEQ ID NO. 94, SEQ ID NO. 95, SEQ ID NO. 96, SEQ ID NO. 97, SEQ ID NO. 98, SEQ ID NO. 99, SEQ ID NO. 100, SEQ ID NO. 101, SEQ ID NO. 102, SEQ ID NO. 103, SEQ ID NO. 104, SEQ ID NO. 105, SEQ ID NO. 106, SEQ ID NO. 107, SEQ ID NO. 108, SEQ ID NO. 109, SEQ ID NO. 110, SEQ ID NO. 111, SEQ ID NO. 112, SEQ ID NO. 113, SEQ ID NO. 114, SEQ ID NO. 115, SEQ ID NO. 116, SEQ ID NO. 117, SEQ ID NO. 118, SEQ ID NO. 119, SEQ ID NO. 120, SEQ ID NO. 121, SEQ ID NO. 122, SEQ ID NO. 123, SEQ ID NO. 124, SEQ ID NO. 125, SEQ ID NO. 126, SEQ ID NO. 127, SEQ ID NO. 128, SEQ ID NO. 129, SEQ ID NO. 130, SEQ ID NO. 131, SEQ ID NO. 132, SEQ ID NO. 133, SEQ ID NO. 134, SEQ ID NO. 135, SEQ ID NO. 136, SEQ ID NO. 137, SEQ ID NO. 138, SEQ ID NO. 139, SEQ ID NO. 140, SEQ ID NO. 141, SEQ ID NO. 142, SEQ ID NO. 143, SEQ ID NO. 144, SEQ ID NO. 145, SEQ ID NO. 146, SEQ ID NO. 147, SEQ ID NO. 148, SEQ ID NO. 149, SEQ ID NO. 150, SEQ ID NO. 151, SEQ ID NO. 152, SEQ ID NO. 153, SEQ ID NO. 154, SEQ ID NO. 155, SEQ ID NO. 156, SEQ ID NO. 157, SEQ ID NO. 158, SEQ ID NO. 159, SEQ ID NO. 160, SEQ ID NO. 161, SEQ ID NO. 162, SEQ ID NO. 163, SEQ ID NO. 164, SEQ ID NO. 165, SEQ ID NO. 166, SEQ ID NO. 167, SEQ ID NO. 168, SEQ ID NO. 169, SEQ ID NO. 170, SEQ ID NO. 171, SEQ ID NO. 172, SEQ ID NO. 173, SEQ ID NO. 174, SEQ ID NO. 175, SEQ ID NO. 176, SEQ ID NO. 177, SEQ ID NO. 178, SEQ ID NO. 179, SEQ ID NO. 180, SEQ ID NO. 181, SEQ ID NO. 182, SEQ ID NO. 183, SEQ ID NO. 184, SEQ ID NO. 185, SEQ ID NO. 186, SEQ ID NO. 187, SEQ ID NO. 188, SEQ ID NO. 189, SEQ ID NO. 190, SEQ ID NO. 191, SEQ ID NO. 192, SEQ ID NO. 193, SEQ ID NO. 194, SEQ ID NO. 195, SEQ ID NO. 196, SEQ ID NO. 197, SEQ ID NO. 198, SEQ ID NO. 199, SEQ ID NO. 200, SEQ ID NO. 201, SEQ ID NO. 202, SEQ ID NO. 203, SEQ ID NO. 204, SEQ ID NO. 205, SEQ ID NO. 206, SEQ ID NO. 207, SEQ ID NO. 208, SEQ ID NO. 209, SEQ ID NO. 210, SEQ ID NO. 211, SEQ ID NO. 212, SEQ ID NO. 213, SEQ ID NO. 214, SEQ ID NO. 215, SEQ ID NO. 216, SEQ ID NO. 217, SEQ ID NO. 218, SEQ ID NO. 219, SEQ ID NO. 220, SEQ ID NO. 221, SEQ ID NO. 222, SEQ ID NO. 223, SEQ ID NO. 224, SEQ ID NO. 225, SEQ ID NO. 226, SEQ ID NO. 227, SEQ ID NO. 228, SEQ ID NO. 229, SEQ ID NO. 230, SEQ ID NO. 231, SEQ ID NO. 232, SEQ ID NO. 233, SEQ ID NO. 234, SEQ ID NO. 235, SEQ ID NO. 236, SEQ ID NO. 237, SEQ ID NO. 238, SEQ ID NO. 239, SEQ ID NO. 240, SEQ ID NO. 241, SEQ ID NO. 242, SEQ ID NO. 243, SEQ ID NO. 244, SEQ ID NO. 245, SEQ ID NO. 246, SEQ ID NO. 247, SEQ ID NO. 248, SEQ ID NO. 249, SEQ ID NO. 250, SEQ ID NO. 251, SEQ ID NO. 252, SEQ ID NO. 253, SEQ ID NO. 254, SEQ ID NO. 255, SEQ ID NO. 256, SEQ ID NO. 257, SEQ ID NO. 258, SEQ ID NO. 259, SEQ ID NO. 260, SEQ ID NO. 261, SEQ ID NO. 262, SEQ ID NO. 263, SEQ ID NO. 264, SEQ ID NO. 265, SEQ ID NO. 266, SEQ ID NO. 267, SEQ ID NO. 268, SEQ ID NO. 269, SEQ ID NO. 270, SEQ ID NO. 271, SEQ ID NO. 272, SEQ ID NO. 273, SEQ ID NO. 274, SEQ ID NO. 275, SEQ ID NO. 276, SEQ ID NO. 277, SEQ ID NO. 278, SEQ ID NO. 279, SEQ ID NO. 280, SEQ ID NO. 281, SEQ ID NO. 282, SEQ ID NO. 283, SEQ ID NO. 284, SEQ ID NO. 285, SEQ ID NO. 286, SEQ ID NO. 287, SEQ ID NO. 288, SEQ ID NO. 289, SEQ ID NO. 290, SEQ ID NO. 291, SEQ ID NO. 292, SEQ ID NO. 293, SEQ ID NO. 294, SEQ ID NO. 295, SEQ ID NO. 296, SEQ ID NO. 297, SEQ ID NO. 298, SEQ ID NO. 299, SEQ ID NO. 300, SEQ ID NO. 301, SEQ ID NO. 302, SEQ ID NO. 303, SEQ ID NO. 304, SEQ ID NO. 305, SEQ ID NO. 306, SEQ ID NO. 307, SEQ ID NO. 308, SEQ ID NO. 309, SEQ ID NO. 310, SEQ ID NO. 311, SEQ ID NO. 312, SEQ ID NO. 313, SEQ ID NO. 314, SEQ ID NO. 315, SEQ ID NO. 316, SEQ ID NO. 317, SEQ ID NO. 318, SEQ ID NO. 319, SEQ ID NO. 320, SEQ ID NO. 321, SEQ ID NO. 322, SEQ ID NO. 323, SEQ ID NO. 324, SEQ ID NO. 325, SEQ ID NO. 326, SEQ ID NO. 327, SEQ ID NO. 328, SEQ ID NO. 329, SEQ ID NO. 330, SEQ ID NO. 331, SEQ ID NO. 332, SEQ ID NO. 333, SEQ ID NO. 334, SEQ ID NO. 335, SEQ ID NO. 336, SEQ ID NO. 337, SEQ ID NO. 338, SEQ ID NO. 339, SEQ ID NO. 340, SEQ ID NO. 341, SEQ ID NO. 342, SEQ ID NO. 343, SEQ ID NO. 344, SEQ ID NO. 345, SEQ ID NO. 346 and SEQ ID NO. 347.

2. (canceled)

3. The Fc variant as claimed in claim 1, having higher binding affinity towards FcRn at pH 6.0 as compared to its said affinity at neutral pH.

4. The Fc variant as claimed in claim 1, having a K_{D} of 10⁻⁸M or less, more preferably 10⁻¹⁰ M or less for FcRn.

5. The Fc variant as claimed in claim 1, that cross-reacts with FcRn from species other than human.

6. The Fc variant as claimed in claim 1 is present in Fc protein of IgG₁, IgG₂, IgG₃, IgG₄ or IgG₂/G₄ isotype, preferably the IgG₁ isotype.

7. The Fc variant as claimed in claim 1 which is expressed in multimer form to increase half-life by increasing the molecular size and affinity through higher avidity of the Fc variant.

8. The multimeric form as claimed in claim 7 is selected from dimer, trimer, tetramer, pentamer and hexamer.

9. The Fc variant as claimed in claim 1 is present in full-length antibody form.

10. The Fc variant as claimed in claim 1 has altered affinity for an Fc gamma receptor relative to the affinity of a wild-type IgG₁ Fc region for the said Fc gamma receptor.

11. The Fc variant as claimed in claim 10, has increased affinity for FcγRIIIa (CD16a) relative to the affinity of a wild-type IgG₁ Fc region for FcγRIIIa including allotypes V158 and F158.

12. The Fc variant as claimed in claim 11, comprising at least one of the following characteristic(s):
a) has an increased half-life in subject;
b) has reduced/no ADCC activity relative to wild-type Fc protein;
c) inhibits phagocytosis mediated by FcγRs and
d) inhibits cytokine release mediated by FcγRs.

13. The Fc variant as claimed in claim 1 is fused to a drug or a therapeutic peptide or polyethylene glycol or an immunogen or neutralizing antibody.

14. The Fc variant as claimed in claim 1 comprises an afucosylated N-linked glycan at EU position 297.

15. The Fc variant as claimed in claim 1 comprising an amino acid sequence selected from sequences as set forth in SEQ ID NO. 1, SEQ ID NO. 2, SEQ ID NO. 3, SEQ ID NO. 4, SEQ ID NO. 5, SEQ ID NO. 7, SEQ ID NO. 9, SEQ ID NO. 11, SEQ ID NO. 14, SEQ ID NO. 15, SEQ ID NO. 18, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 29, SEQ ID NO. 30, SEQ ID NO. 31, SEQ ID NO. 32, SEQ ID NO. 34, SEQ ID NO. 35, SEQ ID NO. 36, SEQ ID NO. 38, SEQ ID NO. 39, SEQ ID NO. 40, SEQ ID NO. 41, SEQ ID NO. 42, SEQ ID NO. 43, SEQ ID NO. 44, SEQ ID NO. 45, SEQ ID NO. 46, SEQ ID NO. 47, SEQ ID NO. 48 and SEQ ID NO. 49.

16. The Fc variant as claimed in claim 1 comprising an amino acid sequence selected from sequences as set forth in SEQ ID NO. 3, SEQ ID NO. 5, SEQ ID NO. 10, SEQ ID NO. 20 or SEQ ID NO. 22.

17. The Fc variant as claimed in claim 16, wherein SEQ ID NO. 22 comprising substitutions T307N, V308P, L309Y, H433R and N434W.

18. The Fc variant as claimed in claim 17, wherein SEQ ID NO. 22 has amino acid sequence selected from SEQ ID NO. 22a, SEQ ID NO. 22b, SEQ ID NO. 22c, SEQ ID NO. 22d SEQ ID NO. 22e and SEQ ID NO. 22f and SEQ ID NO. 22g.

19. A composition comprising Fc variant of any preceding claim and an acceptable carrier.

20. The Fc variant as claimed in any preceding claim is for use in the preparation of a drug either for treating diseases where activity of FcRn is detrimental or for increasing the circulating half-life of a drug or for targeting the drug to certain cells or tissues.

21. The Fc variant as claimed in claim 20 wherein disease is selected from infections, cancer and auto immune disorder.
